(19) United States
(12) Patent Application Publication
(10) Pub. No.: US 2006/0148749 A1
(43) Pub. Date: Jul. 6, 2006

(54) Novel treatment of neurodegenerative diseases by altering levels of TrkB isoforms and/or TrkC isoforms

(75) Inventors: Bruce K. Krueger, Ellicott City, MD (US); Tami J. Kingsbury, Baltimore, MD (US); Linda L. Bambrick, Baltimore, MD (US); Susan G. Dorsey, Frederick, MD (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: University of Maryland, Baltimore, MD (US)

(21) Appl. No.: 11/326,389

(22) Filed: Jan. 6, 2006

Related U.S. Application Data
(63) Continuation of application No. 10/645,546, filed on Aug. 22, 2003.

(30) Foreign Application Priority Data
Feb. 22, 2002 (WO) PCT/US02/05151
May 28, 2002 (WO) PCT/US02/16807

(51) Int. Cl. A61K 48/00

(52) U.S. Cl. 514/44

(57) ABSTRACT

This invention relates to a method of treating or preventing neuro-degenerative disorders and neuro-developmental disorders by altering the ratio of the amount of full-length TrkB polypeptide to the amount of truncated TrkB polypeptides in a neuron or by altering the ratio of the amount of full-length TrkC polypeptide to the amount of truncated TrkC polypeptides in a neuron.

### CROSS REFERENCE TO RELATED APPLICATIONS

This application claims priority under 35 U.S.C. §§ 119 and/or 365 to PCT/US02/16807, filed on May 28, 2002; PCT/US02/05151 filed on Feb. 22, 2002; and to U.S. Provisional Application No. 60/270,553 filed on Feb. 22, 2001, the entire contents of which are hereby incorporated by reference in their entireties for all purposes.

This research was funded in part by grants from the NIH (grant numbers AG10686 and NS40491). The federal government has certain rights to this invention.

### BACKGROUND OF THE INVENTION

1. Field of Invention

This invention relates to a method for treating or preventing neuro-degenerative disorders and neuro-developmental disorders such as Alzheimer's disease, Parkinson's disease, Huntington's disease, amyotrophic lateral sclerosis (Lou Gehrig's disease) and the adverse neurologic complications of Down syndrome, as well as neuron death resulting from injury such as stroke, cerebral ischemia, or chemical and/or physical trauma to the central or peripheral nervous system. This invention further relates to the method of increasing the amount of the full-length TrkB isoform polypeptide in neurons to treat or prevent neuro-degenerative disorders and adverse neurologic complications of Down syndrome. This invention also relates to the method of decreasing the amount of the truncated TrkB isoform polypeptide in neurons to treat or prevent neuro-degenerative disorders, as well as the adverse neurologic complications of Down syndrome.

2. Description of the Related Art

Neurotrophins comprise a class of polypeptide neuron survival factors that not only support the survival of post-mitotic neurons (Lewin and Barde, Physiology of the neurotrophins; Ann. Rev. Neurosci. 19:289-317 (1996)), but also regulate other neuronal functions, including, among others, axon growth and synaptic plasticity (Black IB, Trophic regulation of synaptic plasticity; J. Neurobiol. 41:108-118 (1999); Lentz; et al., Neurotrophins support the development of diverse sensory axon morphologies; J. Neurosci. 19:1038-1048 (1999); Lu and Chow, Neurotrophins and hippocampal synaptic transmission and plasticity; J. Neurosci. Res. 58:76-87 (1999); McAllister et al., Neurotrophins and synaptic plasticity, Ann. Rev. Neurosci. 22:295-318 (1999); Schinder and Poo, The neurotrophin hypothesis for synaptic plasticity, Trends Neurosci. 23:639-645 (2000); Thoenen, Neurotrophins and activity-dependent plasticity, Prog. Brain Res. 128:183-191 (2000)). The class of neurotrophins includes, but is not limited to, nerve growth factor (NGF), brain derived neurotrophic factor (BDNF), neurotrophin-3 (NT-3), and neurotrophin-4/5 (NT4/5). Neurotrophins bind to receptors and activate tyrosine receptor kinases (trks) (Barbacid, The Trk family of neurotrophin receptors, J. Neurobiol. 25:1386-1403 (1994); Bothwell, Functional interactions of neurotrophins and neurotrophin receptors, Ann. Rev. Neurosci. 18:223-253 (1995)). NGF primarily acts via TrkA; BDNF and NT4/5 primarily via TrkB; and NT-3 primarily via TrkC. However the specificity of these interactions are not absolute. Binding of neurotrophins to trk dimers initiates trans auto-phosphorylation of specific tyrosine residues on the intracellular domain of the receptor (Segal and Greenberg, Intracellular signaling pathways activated by neurotrophic factors, Ann. Rev. Neurosci. 19:463-489 (1996); Kaplan and Miller, Neurotrophin signal transduction in the nervous system, Curr. Opinion Neurobiol. 10:381-391 (2000)). These phospho-tyrosine residues serve as docking sites for elements of intracellular signaling cascades that lead to the suppression of neuron death and other effects of the neurotrophins. TrkB and TrkC are also present as truncated forms which lack the intracellular kinase domain and are, therefore, incapable of normal phosphorylation (Klein et al., The trkB tyrosine protein kinase gene codes for a second neurogenic receptor that lacks the catalytic kinase domain, Cell 61:647-656 (1990); Middlemas et al., trkB, a neural receptor protein-tyrosine kinase: evidence for a full-length and two truncated receptors, Mol. Cell Biol. 11:143-153 (1991); Tsoulfas et al., The rat trkC locus encodes multiple neurogenic receptors that exhibit differential response to neurotrophin-3 in PC12 cells, Neuron 10:975-990 (1993)). The full-length and truncated trk isoforms are generated by alternative splicing of the primary trk RNA. While there is some evidence that activation of truncated trk receptors can elicit cellular responses independently of normal tyrosine phosphorylation (Baxter et al., Signal transduction mediated by the truncated trkB receptor isoforms, trkB.T1 and trkB.T2, J. Neurosci. 17:2683-2690 (1997); Hapner et al., Neural differentiation promoted by truncated trkC receptors in collaboration with p75(NTR), Dev. Biol. 201:90-100 (1998); Haapasoalo et al., Expression of the naturally occurring truncated trkB neurotrophin receptor induces outgrowth of filopodia and processes in neuroblastoma cells, Oncogene 18:1285-1296 (1999)), truncated trk receptors are generally thought to inhibit trk-mediated neurotrophin signaling by interacting with full-length receptors to form inactive heterodimers (Eide et al., Neurotrophins and their receptors-current concepts and implications for neurological disease, Exp. Neurol. 121:200-214 (1996)). The expression of truncated trk receptors is developmentally regulated (Fryer et al., Developmental and mature expression of full-length and truncated trkB receptors in the rat forebrain, J. Comp. Neurol. 374:21-40 (1996)) and may represent a normal mechanism for modulating the cellular response to specific neurotrophins (Ninkina et al., Expression and function of TrkB variants in developing sensory neurons, EMBO J. 15:6385-6393 (1996)).

The trisomy 16 (Ts16) mouse has a triplication of chromosome 16 (Coyle et al., Down syndrome, Alzheimer's disease and the trisomy 16 mouse, Trends Neurosci. 11:390-394 (1988)). A cassette of approximately 185 genes on human chromosome 21 is located on mouse chromosome 16 (Hattori et al., The chromosome 21 mapping and sequencing consortium (2000) The DNA sequence of human chromosome 21, Nature 405:311-319 (2000)). As such Ts16 mice share a common genetic defect with the human disorder, Down syndrome (trisomy 21; DS) even though some mouse chromosome 16 genes that are not on human chromosome 21 are overexpressed in Ts16 mice. DS is characterized by mental retardation and, in patients over 40 years of age, Alzheimer's disease (AD) (Mann et al., Alzheimer's presenile dementia, senile dementia of Alzheimer type and Down's syndrome in middle age form an age related continuum of pathological changes, Neuropathol. Appl. Neurobiol. 10:185-207 (1984)). Neurons from embryonic Ts16 mice undergo accelerated death by apoptosis (Bambrick et al., Glutamate as a hippocampal neuron survival factor: an inherited defect in the trisomy 16 mouse, Proc. Natl. Acad. Sci. USA 92:9692-9696 (1995); Stabel-Burow et al., Glutathione levels and nerve cell loss in hippocampal cultures from trisomy 16 mouse—a model of Down syndrome, Brain Res. 765:313-318 (1997); Hallam and Maroun, Anti-gamma interferon can prevent the premature death of trisomy 16 mouse cortical neurons in culture, Neurosci. Lett. 252:17-20 (1998); Bambrick and Krueger, Neuronal apoptosis in mouse trisomy 16: mediation by caspases, J. Neurochem. 72:1769-1772 (1999)), as do cultured cortical neurons from DS fetuses (Busciglio and Yankner, Apoptosis and increased generation of reactive oxygen species in Down's syndrome neurons in vitro, Nature 378:776-779 (1995)). CNS neurons produce BDNF in response to excitatory stimuli. This endogenously produced BDNF mediates activity-dependent neuron survival (Ghosh et al., Requirement for BDNF in activity-dependent survival of cortical neurons, Science 263:1618-1623 (1994)) However, Ts16 hippocampal neurons do not exhibit activity-dependent survival (Bambrick et al., Glutamate as a hippocampal neuron survival factor: an inherited defect in the trisomy 16 mouse, Proc. Natl. Acad. Sci. USA 92:9692-9696 (1995)). It is possible that the accelerated death of Ts16 neurons results from failure of BDNF signaling.

This invention demonstrates that Ts16 neurons fail to respond to BDNF. This failure accounts for their accelerated death and results from altered expression of a trkB isoform.

### BRIEF DESCRIPTION OF THE INVENTION

It is an object of this invention to treat or prevent neuro-degenerative disorders or neuro-developmental disorders by increasing the amount of full-length TrkB polypeptide in neurons. It is a further object of this invention to treat or prevent Alzheimer's disease, Parkinson's disease, Huntington's disease, amyotrophic lateral sclerosis (Lou Gehrig's disease), the adverse neurologic complications of Down syndrome, diabetic peripheral neuropathy, other types of peripheral neuropathy, and neuron death resulting from injury such as stroke, cerebral ischemia, or chemical and/or physical trauma to the central or peripheral nervous system by increasing the amount of full-length TrkB polypeptide in neurons. It is a further object of this invention to treat or prevent Alzheimer's disease, Parkinson's disease, Huntington's disease, amyotrophic lateral sclerosis (Lou Gehrig's disease), the adverse neurologic complications of Down syndrome, diabetic peripheral neuropathy, other types of peripheral neuropathy, and neuron death resulting from injury such as stroke, cerebral ischemia, or chemical and/or physical trauma to the central or peripheral nervous system by increasing the amount of full-length TrkB polypeptide in neurons and by administering neurotrophins. It is another object of this invention that, in order to increase the amount of full-length TrkB polypeptide in neurons, one can administer nucleic acids which encode for full-length TrkB polypeptide or that one can administer full-length TrkB polypeptides.

It is an object of this invention to treat or prevent neuro-degenerative disorders or neuro-developmental disorders by decreasing the amount of truncated TrkB polypeptides in neurons. It is a further object of this invention to treat or prevent Alzheimer's disease, Parkinson's disease, Huntington's disease, amyotrophic lateral sclerosis (Lou Gehrig's disease), the adverse neurologic complications of Down syndrome, diabetic peripheral neuropathy, other types of peripheral neuropathy, and neuron death resulting from injury such as stroke, cerebral ischemia, or chemical and/or physical trauma to the central or peripheral nervous system by decreasing the amount of truncated TrkB polypeptides in neurons. It is also an object of this invention to treat or prevent Alzheimer's disease, Parkinson's disease, Huntington's disease, amyotrophic lateral sclerosis (Lou Gehrig's disease), the adverse neurologic complications of Down syndrome, diabetic peripheral neuropathy, other types of peripheral neuropathy, and neuron death resulting from injury such as stroke, cerebral ischemia, or chemical and/or physical trauma to the central or peripheral nervous system by decreasing the amount of truncated TrkB polypeptides in neurons and by administering neurotrophins. It is a further object of this invention that one can decrease the amount of truncated TrkB polypeptides in neurons by administering nucleic acids which encode anti-sense RNA specific for truncated TrkB polypeptides or by administering nucleic acids which encode for double stranded RNA specific for truncated TrkB polypeptides.

It is an object of this invention to treat or prevent neuro-degenerative disorders or neuro-developmental disorders by increasing the ratio of the amount of full-length TrkB polypeptide to the amount of truncated TrkB polypeptides. It is a further object of this invention to treat or prevent Alzheimer's disease, Parkinson's disease, Huntington's disease, amyotrophic lateral sclerosis (Lou Gehrig's disease), the adverse neurologic complications of Down syndrome, diabetic peripheral neuropathy, other types of peripheral neuropathy, and neuron death resulting from injury such as stroke, cerebral ischemia, or chemical and/or physical trauma to the central or peripheral nervous system by increasing the ratio of the amount of full-length TrkB polypeptide to the amount of truncated TrkB polypeptides. It is also an object of this invention to treat or prevent Alzheimer's disease, Parkinson's disease, Huntington's disease, amyotrophic lateral sclerosis (Lou Gehrig's disease), the adverse neurologic complications of Down syndrome, diabetic peripheral neuropathy, other types of peripheral neuropathy, and neuron death resulting from injury such as stroke, cerebral ischemia, or chemical and/or physical trauma to the central or peripheral nervous system by increasing the ratio of the amount of full-length TrkB polypeptide to the amount of truncated TrkB polypeptides in neurons and by administering neurotrophins. It is a further object of this invention that one can increase the ratio of the amount of full-length TrkB polypeptide to the amount of truncated TrkB polypeptides by administering nucleic acids or polypeptides which encode for full-length TrkB polypeptide or by administering nucleic acids which encode for anti-sense RNA specific for truncated TrkB polypeptides or by administering nucleic acids which encode for double stranded RNA specific for truncated TrkB polypeptides, or by administering a combination thereof.

It is an object of this invention to treat or prevent neuro-degenerative disorders or neuro-developmental disorders by increasing the amount of full-length TrkC polypeptide in neurons. It is a further object of this invention to treat or prevent Alzheimer's disease, Parkinson's disease, Huntington's disease, amyotrophic lateral sclerosis (Lou Gehrig's disease), the adverse neurologic complications of Down syndrome, diabetic peripheral neuropathy, other types of peripheral neuropathy, and neuron death resulting from injury such as stroke, cerebral ischemia, or chemical and/or physical trauma to the central or peripheral nervous system by increasing the amount of full-length TrkC polypeptide in neurons. It is a further object of this invention to treat or prevent Alzheimer's disease, Parkinson's disease, Huntington's disease, amyotrophic lateral sclerosis (Lou Gehrig's disease), the adverse neurologic complications of Down syndrome, diabetic peripheral neuropathy, other types of peripheral neuropathy, and neuron death resulting from injury such as stroke, cerebral ischemia, or chemical and/or physical trauma to the central or peripheral nervous system by increasing the amount of full-length TrkC polypeptide in neurons and by administering neurotrophins. It is another object of this invention that, in order to increase the amount of full-length TrkC polypeptide in neurons, one can administer nucleic acids which encode for full-length TrkB polypeptide or that one can administer full-length TrkC polypeptides.

It is an object of this invention to treat or prevent neuro-degenerative disorders or neuro-developmental disorders by decreasing the amount of truncated TrkC polypeptides in neurons. It is a further object of this invention to treat or prevent Alzheimer's disease, Parkinson's disease, Huntington's disease, amyotrophic lateral sclerosis (Lou Gehrig's disease), the adverse neurologic complications of Down syndrome, diabetic peripheral neuropathy, other types of peripheral neuropathy, and neuron death resulting from injury such as stroke, cerebral ischemia, or chemical and/or physical trauma to the central or peripheral nervous system by decreasing the amount of truncated TrkC polypeptides in neurons. It is also an object of this invention to treat or prevent Alzheimer's disease, Parkinson's disease, Huntington's disease, amyotrophic lateral sclerosis (Lou Gehrig's disease), the adverse neurologic complications of Down syndrome, diabetic peripheral neuropathy, other types of peripheral neuropathy, and neuron death resulting from injury such as stroke, cerebral ischemia, or chemical and/or physical trauma to the central or peripheral nervous system by decreasing the amount of truncated TrkC polypeptides in neurons and by administering neurotrophins. It is a further object of this invention that one can decrease the amount of truncated TrkC polypeptides in neurons by administering nucleic acids which encode for anti-sense RNA specific for truncated TrkC polypeptides or by administering nucleic acids which encode for double stranded RNA specific for truncated TrkC polypeptides.

It is an object of this invention to treat or prevent neuro-degenerative disorders or neuro-developmental disorders by increasing the ratio of the amount of full-length TrkC polypeptide to the amount of truncated TrkC polypeptides. It is a further object of this invention to treat or prevent Alzheimer's disease, Parkinson's disease, Huntington's disease, amyotrophic lateral sclerosis (Lou Gehrig's disease), the adverse neurologic complications of Down syndrome, diabetic peripheral neuropathy, other types of peripheral neuropathy, and neuron death resulting from injury such as stroke, cerebral ischemia, or chemical and/or physical trauma to the central or peripheral nervous system by increasing the ratio of the amount of full-length TrkC polypeptide to the amount of truncated TrkC polypeptides. It is a further object of this invention that one can increase the ratio of the amount of full-length TrkC polypeptide to the amount of truncated TrkC polypeptides by administering nucleic acids which encode for full-length TrkC polypeptide or by administering nucleic acids which encode for anti-sense RNA specific for truncated TrkC polypeptides or by administering nucleic acids which encode for double stranded RNA specific for truncated TrkC polypeptides, or by administering a combination thereof.

It is also an object of this invention to inhibit the progression of a neuro-degenerative disorder or a neuro-developmental disorder in a mammal by administering a vector containing nucleic acids to alter the ratio of the amount of full-length TrkB polypeptide to the amount of truncated TrkB polypeptides in a neuron. It is a further object of this invention that the vector contain isolated nucleic acid encoding (a) full-length TrkB polypeptide, (b) anti-sense RNA specific for truncated TrkB polypeptides, (c) double stranded RNA specific for truncated TrkB polypeptides, or (d) a combination thereof. It is another object of this invention that the vector be a plasmid or a virus, and if a virus, be selected from a group consisting of herpesvirus, adenovirus, adeno associated virus, retrovirus, vaccinia virus, and canary pox virus.

It is another an object of this invention to inhibit the progression of a neuro-degenerative disorder or a neuro-developmental disorder in a mammal by administering a vector containing nucleic acids to alter the ratio of the amount of full-length TrkC polypeptide to the amount of truncated TrkC polypeptides in a neuron. It is a further object of this invention that the vector contain isolated nucleic acid encoding for (a) full-length TrkC polypeptide, (b) anti-sense RNA specific for truncated TrkC polypeptides, (c) double stranded RNA specific for truncated TrkC polypeptides, or (d) a combination thereof. It is another object of this invention that the vector be a plasmid or a virus, and if a virus, be selected from a group consisting of herpesvirus, adenovirus, adeno associated virus, retrovirus, vaccinia virus, and canary pox virus.

It is an object of this invention to treat a disease characterized by an increased ratio of the amount of truncated TrkB polypeptides to the amount of full-length TrkB polypeptides in a cell as compared to the ratio of these polypeptides in a normal, healthy mammal by administering a vector containing nucleic acids to alter the ratio of the amount of truncated TrkB polypeptides to the amount of full-length TrkB polypeptide in a cell. It is a further object of this invention that the vector contain isolated nucleic acid encoding for (a) full-length TrkB polypeptide, (b) anti-sense RNA specific for truncated TrkB polypeptides, (c) double stranded RNA specific for truncated TrkB polypeptides, or (d) a combination thereof. It is another object of this invention that the vector be a plasmid or a virus, and if a virus be selected from a group consisting of herpesvirus, adenovirus, adeno associated virus, retrovirus, vaccinia virus, and canary pox virus.

It is an object of this invention to treat a disease characterized by an increased ratio of the amount of truncated TrkC polypeptides to the amount of full-length TrkC polypeptides in a cell as compared to the ratio of these polypeptides in a normal, healthy mammal by administering a vector containing nucleic acids to alter the ratio of the amount of truncated TrkC polypeptides to the amount of full-length TrkC polypeptide in a cell. It is a further object of this invention that the vector contain isolated nucleic acid encoding for (a) full-length TrkC polypeptide, (b) anti-sense RNA specific for truncated TrkC polypeptides, (c) double stranded RNA specific for truncated TrkC polypeptides, or (d) a combination thereof. It is another object of this invention that the vector be a plasmid or a virus, and if a virus be selected from a group consisting of herpesvirus, adenovirus, adeno associated virus, retrovirus, vaccinia virus, and canary pox virus.

It is another object of this invention to inhibit the progression of a neuro-degenerative disorder or a neuro-developmental disorder in an animal by administering (a) a polypeptide for full-length TrkB, or a mutant, variant, homolog, or fragment thereof having the same activity as full-length TrkB, (b) a polypeptide for full-length TrkC, or a mutant, variant, homolog, or fragment thereof having the same activity as full-length TrkC, (c) a nucleic acid encoding for full-length TrkB, or a mutant, variant, homolog, or fragment thereof having the same activity as full-length TrkB, (d) a nucleic acid encoding for full-length TrkC, or a mutant, variant, homolog, or fragment thereof having the same activity as full-length TrkC, or (e) a combination of the above.

It is an object of this invention to treat or prevent neuro-degenerative disorders or neuro-developmental disorders by administering exogenous polynucleotides which encode full-length TrkB polypeptide to increase the expression of full-length TrkB polypeptide. It is a further object of this invention to administer neurotrophins in combination with the administered exogenous polynucleotides which encode for full-length TrkB polypeptide. It is a further object of this invention that the neuro-degenerative disorders or neuro-developmental disorders Alzheimer's disease, Parkinson's disease, Huntington's disease, amyotrophic lateral sclerosis (Lou Gehrig's disease), the adverse neurologic complications of Down syndrome, diabetic peripheral neuropathy, and other types of peripheral neuropathy. It is also an object of this invention that neuro-degenerative disorders or neuro-developmental disorders can include neuron death resulting from an injury such as a stroke, cerebral ischemia, or chemical and/or physical trauma; to the central or peripheral nervous system.

It is an object of this invention to treat or prevent neuro-degenerative disorders or neuro-developmental disorders by administering exogenous polynucleotides to decrease the expression of truncated TrkB polypeptides. It is a further object of this invention to administer neurotrophins in combination with the administered exogenous polynucleotides. It is also an object of this invention that the exogenous polynucleotides encode for anti-sense RNA or double stranded RNA for truncated trkb. It is a further object of this invention that the neuro-degenerative disorders or neuro-developmental disorders Alzheimer's disease, Parkinson's disease, Huntington's disease, amyotrophic lateral sclerosis (Lou Gehrig's disease), the adverse neurologic complications of Down syndrome, diabetic peripheral neuropathy, and other types of peripheral neuropathy. It is also an object of this invention that neuro-degenerative disorders or neuro-developmental disorders can include neuron death resulting from an injury such as a stroke, cerebral ischemia, or chemical and/or physical trauma; to the central or peripheral nervous system.

It is an object of this invention to treat or prevent neuro-degenerative disorders or neuro-developmental disorders by administering exogenous polynucleotides which encode for full-length TrkC polypeptide to increase the expression of full-length TrkC polypeptide. It is a further object of this invention to administer neurotrophins in combination with the administered exogenous polynucleotides which encode for full-length TrkC polypeptide. It is a further object of this invention that the neuro-degenerative disorders or neuro-developmental disorders Alzheimer's disease, Parkinson's disease, Huntington's disease, amyotrophic lateral sclerosis (Lou Gehrig's disease), the adverse neurologic complications of Down syndrome, diabetic peripheral neuropathy, and other types of peripheral neuropathy. It is also an object of this invention that neuro-degenerative disorders or neuro-developmental disorders can include neuron death resulting from an injury such as a stroke, cerebral ischemia, or chemical and/or physical trauma; to the central or peripheral nervous system.

It is an object of this invention to treat or prevent neuro-degenerative disorders or neuro-developmental disorders by administering exogenous polynucleotides to decrease the expression of truncated TrkC polypeptides. It is a further object of this invention to administer neurotrophins in combination with the administered exogenous polynucleotides. It is also an object of this invention that the exogenous polynucleotides encode for anti-sense RNA or double stranded RNA for truncated trkC. It is a further object of this invention that the neuro-degenerative disorders or neuro-developmental disorders Alzheimer's disease, Parkinson's disease, Huntington's disease, amyotrophic lateral sclerosis (Lou Gehrig's disease), the adverse neurologic complications of Down syndrome, diabetic peripheral neuropathy, and other types of peripheral neuropathy. It is also an object of this invention that neuro-degenerative disorders or neuro-developmental disorders can include neuron death resulting from an injury such as a stroke, cerebral ischemia, or chemical and/or physical trauma; to the central or peripheral nervous system.

It is an object of this invention to have a pharmaceutical composition containing a vector having nucleic acids encoding for full-length TrkB polypeptide; and a pharmaceutically acceptable carrier.

It is another object of this invention to have a pharmaceutical composition containing a vector having nucleic acids encoding for full-length TrkC polypeptide; and a pharmaceutically acceptable carrier.

It is another object of this invention to have a pharmaceutical composition containing a vector having nucleic acids encoding for anti-sense RNA or double stranded RNA for a truncated TrkB isoform; and a pharmaceutically acceptable carrier.

It is another object of this invention to have a pharmaceutical composition containing a vector having nucleic acids encoding for anti-sense RNA or double stranded RNA for a truncated TrkC isoform; and a pharmaceutically acceptable carrier.

### BRIEF DESCRIPTION OF THE FIGURES

FIG. 1A illustrates the survival of euploid (filled bars) and Ts16 (open bars) hippocampal neurons at 5.5 days in vitro in the continuous presence of B27.

FIG. 1B shows the survival of euploid (filled bars) and Ts16 (open bars) neurons 16 hours after withdrawal of B27 at 3 days in vitro.

FIG. 2A shows the abnormal expression of TrkB isoforms in Ts16 neurons (Ts) and normal (eu) neurons via western blot, where the full-length isoform is at 145 and the truncated isoform is at 95.

FIG. 2B illustrates the ratio of TrkB.FL to TrkB.T1 in euploid and Ts16 neurons.

FIG. 2C illustrates a western blot of euploid neurons (eu) and Ts16 neurons (Ts) using anti-TrkB(T1) which labels an internal epitope on TrkB.T1. The band appears at 95.

FIG. 2D shows a western blot of euploid neurons (eu) and Ts16 neurons (Ts) using anti-p75, having a band at 75.

FIG. 2E shows a western blot of euploid neurons (eu) and Ts16 neurons (Ts) using an antibody to TrkC that labels both the full length isoform (150 kDa) and the truncated isoform (110 kDa).

FIG. 3A is a western blot showing the level of expression of exogenous TrkB.T1 in euploid neurons exposed to adenovirus carrying TrkB.T1-HA DNA (AdTR) and euploid neurons exposed to an adenovirus control (Ad-).

FIG. 3B shows a western blot showing the level of expression of exogenous TrkB.FL in Ts16 neurons exposed to adenovirus carrying TrkB.FL-HA DNA (AdFL) and Ts16 neurons exposed to an adenovirus control (Ad-).

FIG. 3C illustrates the survival of neurons infected with adenovirus control (Ad-) (▾), adenovirus carrying TrkB.FL-HA DNA (AdFL) (∇), and adenovirus carrying TrkB.T1-HA DNA (AdTR) (∘), and untreated neurons (•). The expression of TrkB.T1 in euploid neurons inhibits BDNF survival signaling.

FIG. 3D illustrates the survival of Ts16 neurons infected with adenovirus control (Ad-) (▾), adenovirus carrying TrkB.FL-HA DNA (AdFL) (v), and adenovirus carrying TrkB.T1-HA DNA (AdTR) (∘), and untreated neurons (•). The expression of TrkB.FL in Ts16 neurons neurons restores BDNF survival signaling.

FIG. 3E summarizes the effect of TrkB.FL expression on BDNF survival signaling; the survival of euploid neurons (with and with BDNF treatment), Ts16 neurons (with and without BDNF treatment), and Ts16 neurons (with and without BDNF treatment) is shown.

### DETAILED DESCRIPTION OF THE INVENTION

This invention involves using gene therapy to treat or prevent neuro-degenerative disorders and developmental disorders such as Alzheimer's disease (AD), Parkinson's disease (PD), Huntington's disease (HD), amyotrophic lateral sclerosis (Lou Gehrig's disease) (ALS) and the adverse neurologic complications of Down syndrome (DS). For the purposes of this invention, neuro-degenerative disorders and developmental disorders can include neural apoptosis or death resulting from injury where the injury can include, but not be limited to, stroke, cerebral ischemia, or chemical and/or physical trauma to the central or peripheral nervous system. Furthermore, this invention involves using nucleic acids encoding the full-length isoforms of TrkB and TrkC, the truncated isoforms of TrkB and TrkC, anti-sense RNA against the full length and truncated isoforms TrkB, and anti-sense RNA against the full-length and truncated isoforms of TrkC to treat or prevent neuro-degenerative disorders and developmental disorders. One utilizes these nucleic acids to preferentially express in a desired cell a desired nucleic acid or a desired nucleic acid and its encoded polypeptide to alter the level of endogenous expression of the isoforms of TrkB and/or the isoforms of TrkC. This invention also involves using polypeptides for full length TrkB and/or full length TrkC to treat or prevent neuro-degenerative disorders and developmental disorders. One can alter the ratio of the amount of truncated TrkB to full length TrkB in a cell, or the ratio of the amount of truncated TrkC to full length TrkC, or the ratio of full length TrkB to truncated TrkC, or the ratio of full length TrkC to truncated TrkB in a cell to order to treat or prevent the above mentioned neuro-degenerative disorders and developmental disorders.

In addition, this invention involves using nucleic acids encoding the full-length isoforms of TrkB and TrkC, the truncated isoforms of TrkB and TrkC, anti-sense RNA against the full length and truncated isoforms TrkB, and anti-sense RNA against the full-length and truncated isoforms of TrkC to selectively induce neural apoptosis.

Increasing the level of expression of full-length TrkB polypeptide or decreasing the level of expression of truncated TrkB polypeptide is shown herein to protect Ts16 hippocampal neurons from death when exposed to BDNF. Furthermore, increasing the level of expression of full-length TrkB polypeptide or decreasing the level of expression of truncated TrkB polypeptide in mouse Ts16 neurons, a naturally occurring model for DS, resulted in a slower rate of apoptosis when the neurons are exposed to BDNF, demonstrating the anti-apoptotic activity of alterations of the level of expression of the truncated and full-length versions of TrkB specifically with respect to genetic defects associated with neurodegeneration. Given that many clinically-significant neuro-degenerative disorders are characterized by neuronal apoptosis, the invention makes use of the anti-apoptotic activity of altered levels of expression of truncated and full-length TrkB polypeptides to treat such disorders, including, but not limited to, AD, ALS, DS, PD, and HD. The data presented herein demonstrate the usefulness of altering the levels of expression of full-length and truncated TrkB polypeptides in inhibiting neuronal apoptosis, including that associated with neuro-degenerative disorders.

The invention includes a method of inhibiting apoptosis of neuronal cells in a mammal. The method comprises administering to the mammal an apoptosis-inhibiting amount of an isolated nucleic acids encoding full-length TrkB, anti-sense RNA specific for one or more isoforms of truncated TrkB, double-stranded RNA specific for one or more isoforms of truncated TrkB, full-length TrkC, anti-sense RNA specific for one or more isoforms of truncated TrkC, and/or double-stranded RNA specific for one or more isoforms of truncated TrkC.

For this invention, the amino acid and nucleotide sequences of the human full-length TrkB, human truncated TrkB isoforms (for example, TrkB.T1 and TrkB.Shc), mouse full-length TrkB, and mouse truncated TrkB isoforms (for example, TrkB.T1) are useful Also useful for this invention are the amino acid and nucleotide sequences of the human full-length TrkC, human truncated TrkC isoforms, mouse full-length TrkC, and mouse truncated TrkC isoforms.

The human full-length TrkB nucleotide sequence (SEQ ID NO: 1) and amino acid sequence (SEQ ID NO: 2) are found at GenBank accession number NM_{—}006180. Recently, it was reported that there are multiple distinct isoforms of truncated TrkB (Stoilov P, et al., Analysis of the Human TrkB Gene Genomic Organization Reveals Novel TrkB Isoforms, Unusual Gene Length, and Splicing Mechanism, Biochem. Biophys. Res. Commun., 290(3): 1054-1065 (2002)). One isoform is a homolog of the mouse truncated TrkB.T1 and the other isoform, designated TrkB.Shc. TrkB.Shc contains a tyrosine that binds to the downstream effector, shc, but lacks kinase activity. In fact, it has been report that there are at least two isoforms of the human TrkB.Shc. The nucleotide sequence (SEQ ID NO: 3) and the amino acid sequence (SEQ ID NO: 4) for the human homolog of mouse TrkB.T1 are found at GenBank accession number S76474. The nucleotide sequence (SEQ ID NO: 5) and the amino acid sequence (SEQ ID NO: 6) for one isoform of human TrkB.Shc are found at GenBank accession number AF410900. The nucleotide sequence (SEQ ID NO: 7) and the amino acid sequence (SEQ ID NO: 8) for the other isoform of human TrkB.Shc are found at GenBank accession number AF410901.

The nucleotide sequence (SEQ ID NO: 9) and amino acid sequence (SEQ ID NO: 10) for the mouse full-length TrkB (TrkB.FL) are found at GenBank accession number X17647. The nucleotide sequence (SEQ ID NO: 11) and amino acid sequence (SEQ ID NO: 12) for the mouse truncated TrkB (TrkB.T1) are found at GenBank accession number M33385.

The human full-length TrkC nucleotide sequence (SEQ ID NO: 13) and amino acid sequence (SEQ ID NO: 14) are found at GenBank accession number XM_{—}038336. Human truncated TrkC nucleotide sequences for two exons (exons 13B and 14B) which are specific for this protein are listed with GenBank. The nucleotide sequence for exon 13B (SEQ ID NO: 15) is found at GenBank accession numbers AJ224536 and the nucleotide sequence for exon 14B (SEQ ID NO: 16) is found at GenBank accession numbers AJ224537.

It appears that there are two isoforms of truncated mouse TrkC (isoform 1 and isoform 2). For isoform 1 of mouse truncated TrkC, the nucleotide and amino acid sequences are found at GenBank accession number AF035399. For isoform 2 of mouse truncated TrkC, the nucleotide and amino acid sequences are found at GenBank accession number AF035400.

Also useful to the invention is an isolated full-length TrkB polypeptide or a mutant, variant, homolog, or fragment thereof having the activity of full-length TrkB, as described herein.

Useful to the invention is an isolated full-length TrkC polypeptide or a mutant, variant, homolog, or fragment thereof having the activity of full-length TrkC, as described herein.

Also useful in this invention is anti-sense RNA specific for the various proteins of this invention (e.g., isoforms of truncated TrkB, isoforms of truncated TrkC, full-length TrkB, and full-length TrkC) and polynucleotides which encode the anti-sense RNA. Anti-sense RNA can range in size from 10 through 100, more preferably from 18 through 30, nucleotides long, if the anti-sense RNA is being administered directly to a cell. If, however, the anti-sense RNA is to generated inside a cell using a vector, the coding sequences for the anti-sense RNA can range from 20 to several thousand nucleotides in length.

One example the anti-sense RNA specific for mouse truncated TrkB.T1 is the 1089 base pair sequence in SEQ ID NO: 17. Another example of anti-sense RNA sequence useful for reducing the amount of mouse truncated TrkB in a cell is AAGCAGGCUG CAGACAUCCU (SEQ ID NO: 18). An example of anti-sense RNA useful for reducing the amount of human truncated TrkB.T1 in a cell is provided in SEQ ID NO: 19. An example of anti-sense RNA useful for reducing the amount of human truncated TrkB.Shc in a cell is provided in SEQ ID NO: 20; this sequence is directed at exon 19 which appears to be conserved among the isoforms of TrkB.Shc. For all anti-sense RNA sequences, one can replace thymine with uracil or replace uracil with thymine.

Two examples of anti-sense RNA specific for human truncated TrkC are provided. One sequence (SEQ ID NO: 21) is specific for exon 13B; the other sequence (SEQ ID NO: 22) is specific for exon 14B. Alternatively, one can use both sequences in tandem to generate an anti-sense RNA specific for exons 13B and 14B of human truncated TrkC.

Double-stranded RNA specific for the various proteins of this invention (e.g., isoforms of truncated TrkB, isoforms of truncated TrkC, full-length TrkB, and full-length TrkC) and polynucleotides which encode the double-stranded RNA are also useful in this invention. With double-stranded RNA, one can generate double-stranded RNA having lengths of 10, 15, 20, 25, 30, 35, 40, 45, 50, or more base pairs. It is preferable that these double-stranded RNA are specific for the unique sequences for the gene for which one is trying to inhibit transcription or translation. For human TrkB.T1, one can use double-stranded RNA for any of the sequences listed in SEQ ID NO: 19; for human TrkB.Shc, use sequences in SEQ ID NO: 20; for human TrkC use sequences in SEQ ID NO: 21 or SEQ ID NO: 22.

A number of TrkB and TrkC encoding nucleic acid combinations are useful in the invention. For example, an isolated nucleic acid encoding full-length TrkB may be delivered to a neuron in combination with an isolated nucleic acid encoding full-length TrkC. In another example, anti-sense RNA specific for one or more isoforms of truncated TrkB and for one or more isoforms of truncated TrkC may be delivered to a neuron in combination with each other. Another example of a combination is nucleic acids encoded for full-length TrkB and for anti-sense RNA specific for one or more isoforms of truncated TrkC. Yet another example is anti-sense RNA specific for one or more isoforms of truncated TrkB and full-length TrkC. Also covered by this invention is the combination of polynucleotides encoding full-length TrkB and anti-sense RNA specific for one or more isoforms of truncated TrkB. Also covered is the combination of polynucleotides encoding full-length TrkC and anti-sense RNA specific for one or more isoforms of truncated TrkC. These combination nucleic acids can be linked using standard molecular biology techniques and delivered as a single fused nucleic acid molecule, or they may be present in distinct and separate plasmids or vectors, or the nucleic acids may be on one plasmid or vector but under the control of different promoters. The nucleic acids can be polycistronic under one promoter, or they can be expressed independently using different promoters. Further, fragments of either molecule may be delivered, wherein each fragment retains biological activity of the respective protein encoded thereby.

### Modes of Administration

The isolated nucleic acid encoding full length TrkB or the isolated nucleic acid encoding for anti-sense truncated TrkB can be administered to a mammal using a variety of methods. In a preferred embodiment of the invention, trkB polynucleotides are delivered using a vector. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, herpesvirus vectors, adenoviral vectors, adeno-associated virus vectors, retroviral vectors, and the like.

Useful in the invention is a vector comprising the nucleic acid encoding TrkB (either anti-sense truncated or sense full length isoform). Also useful is a vector comprising the nucleic acid encoding for TrkC (either anti-sense truncated or sense full length isoform). The nucleic acids may be present within separate vectors or within the same vector. When the nucleic acids are within the same vector, the nucleic acids may be polycistronic such that their expression is linked to one another or they may be expressed independently from one another. Many vectors may be useful for delivering the combination of TrkB and TrkC to cells in a mammal.

Given the neurotropism of Herpes Simplex Virus 2 (HSV-2), this virus serves as a useful vector for delivery of polynucleotides encoding TrkB and/or TrkC (full-length and truncated isoforms) and polynucleotides encoding anti-sense RNA and double-stranded RNA specific for TrkB and/or TrkC(full-length and truncated isoforms) to neurons. Particularly useful in the invention, is an HSV-2 vector wherein the RR domain of ICP10 in HSV-2 have been deleted (ICP10deltaRR), thereby rendering the virus replication-defective but retaining the anti-apoptotic activity of the PK domain of ICP10. Alternatively, one can use a HSV-2 vector where both the RR and PK domains in HSV-2 have been deleted (ICP10deltaPK,RR). Other viral and non-viral vectors containing the desired polynucleotides of this invention may also be useful in the invention. For example, retrovirus vectors containing the desired polynucleotides can be used to stably infect neuronal stem cells useful in ex-vivo gene therapy. Other viral vectors including, but not limited to, adenovirus, vaccinia virus, canary pox virus, and adeno associated virus are useful for this invention.

Vectors containing the desired polynucleotides can be constructed by standard molecular biology techniques. An HSV-2 vector, ICP10deltaRR, wherein the RR domain of ICP10 was replaced with a nucleic acid encoding LacZ was constructed previously (U.S. Pat. Nos. 6,013,265, 6,054,131, and 6,207,168). The addition of polynucleotides encoding for TrkB and/or TrkC isoforms (full-length and truncated), anti-sense RNA specific for TrkB and/or TrkC isoforms (full-length and truncated), and/or double-stranded RNA specific for TrkB and/or TrkC isoforms (full-length and truncated) to this HSV-2 vector can be accomplished using well-known in the art-field techniques. Other HSV-2 vectors encoding the desired polynucleotides of this invention can be constructed by similar methods.

Also useful in the invention is having the desired polynucleotide sequences operably linked to a promoter regulatory sequence that facilitates expression of the desired polynucleotide sequences. Tissue specific and/or inducible promoters particularly useful for this invention. Because the invention relates to the expression of the desired polynucleotide sequences in neuronal cells, the following neuron-specific promoters will be particularly useful: neuron-specific enolase (NSE) and tyrosine hydroxylase (TH) promoters, TH-NFH (neurofilament heavy subunit) chimeric promoter, and the golli promoter (each of these promoters is described in detail below). Endogenous mammalian NSE is expressed in essentially all neurons, beginning during development at the time of synaptogenesis; its activity increases at a steady rate into adulthood when amounts of this protein can reach levels of up to 1% of the total cell protein (Marangos, et al., Neuron specific enolase, a clinically useful marker for neurons and neuroendocrine cells, Ann. Rev. Neurosci. 60:269-295 (1987)). The pattern of expression of this promoter makes it a good candidate for conferring long-term expression of foreign genes on adult neurons following delivery by a viral vector. The TH-NFH promoter supports long-term gene expression in striatal neurons (Wang, et al., General strategy for constructing large HSV-1 plasmid vectors that co-express multiple genes, Biotechniques 31:204-212 (2001)). Golli products of the myelin basic protein (MBP) gene have been found to be expressed in neurons during postnatal and embryonic development including Cajal-Retzius and cortical subplate neurons. Moreover, golli expression occurs in other cortical neurons including neurons from cortical layer V and the hippocampus (Pribyl, et al., Expression of the myelin basic protein gene locus in neurons and oligodendrocytes in the human fetal central nervous system, J. Comp. Neurol. 374:342-353 (1996); Pribyl, et al., The human myelin basic protein gene is included within a 179-kilobase transcription unit: expression in the immune and central nervous systems, Proc. Natl. Acad. Sci. USA 90:10695-10699 (1993)). Consequently, the golli promoter may be useful for driving transgene expression in selected neuronal populations.

Viral promoters including the HSV latency associated transcript (LAT) promoter, the Moloney murine leukemia virus (Mo-MLV) long terminal repeat (LTR), and the human cytomegalovirus (HCMV) immediate early (1E) promoter may also by useful The LAT promoter includes elements both upstream and downstream of the start site of the minor LAT mRNA from which the intranuclear LATs are derived. Promoter elements referred to as LAP2 (latency active promoter 2) and LAP™ (contains neuronal responsive elements) are independently capable of expressing LAT during viral latency in sensory ganglia. The transgene can be placed downstream of LAP1 near the start of the LAT mRNA or downstream of both promoters within the LAT intron. Stable transgene expression has been achieved in sensory ganglia, but expression in CNS neurons was less vigorous (Fink, et al., Engineering herpes simplex virus vectors for gene transfer to neurons, Nature Med. 3:357-359 (1997)). The LTR of Mo-MLV has been used with HSV vectors to yield stable expression of the LacZ gene in sensory neurons and extended expression in motor neurons of the hypoglossal nucleus (Dobson, et al., A latent, nonpathogenic HSV-1-derived vector stably expresses beta-galactosidase in mouse neurons, Neuron 5:353-360 (1990)). The HCMV IE promoter is a very strong constitutive promoter that is active in a wide variety of cell types including CNS neurons both in vitro (Johnson, et al., Effects of gene transfer into cultured CNS neurons with a replication-defective herpes simplex virus type 1 vector, Mol. Brain Res. 12:95-102 (1992)) and in vivo (Wood, et al., Specific patterns of defective HSV-1 gene transfer in the adult central nervous system: implications for gene targeting, Exp. Neurol. 130:127-140 (1994)). The vectors described above may also comprise such promoters operably linked to the desired polynucleotide sequences.

Another useful delivery technique of nucleotides and polypeptides is intracranial injection of the nucleic acids, or of a vector containing the desired nucleic acids, or of the polypeptides. One can also combine polynucleotides with basic polypeptides, such as poly-lysine and poly-histidine, prior to applying and/or injecting the polynucleotides into neurons.

Another useful delivery technique of polynucleotides, including vectors, is electropermeabilization. Electropermeabilization can be used in gene therapy to administer DNA directly to an animal (Drabick, J J, et al., Cutaneous transfection and immune responses to intradermal nucleic acid vaccination are significantly enhanced by in vivo electropermeabilization, Mol. Ther., 3(2):249-55 (2001)). Alternatively, electroporation can be used to get DNA into a cell and then the cell is placed inside the animal. Electroporation is well-known in the art field and can be performed using the following briefly described method: A mixture of 150 ml cells and plasmid DNA are electroporated in a 0.2 cm curettes in a Gene Pulser (BioRad Laboratories, Hercules, Calif.) using 2.5 kV, 200 W, 25 mF, or 1.75 kV, 600 W, 25 mF. The plasmid DNA can encode anti-sense RNA, double-stranded RNA, and/or full-length or truncated proteins under control of a constitutive or inducible promoter, as described above. Combining the polynucleotides with basic polypeptides, such as poly-lysine and poly-histidine, may be useful prior to electropermeabilization or electroporation.

Synthesized oligonucleotides can be introduced into suitable cells by a variety of means including electroporation, calcium phosphate precipitation, or microinjection. Polynucleotides may also be introduced into cells by using bacteria as carriers (see for example U.S. Pat. No. 6,150,170; and International Patent Application PCT/US98/21093 filed Oct. 7, 1998).

In the methods of the invention, full-length or truncated TrkB isoforms may be delivered to neuronal cells in the form of a nucleic acids encoding full-length or truncated TrkB isoforms, preferably using vectors or liposomes, or it may be delivered to cells in the form of a polypeptide, or a mutant, variant, homolog, or fragment thereof having the activity of full-length or truncated TrkB isoforms using liposomes. Thus, the use of full-length or truncated TrkB isoform polypeptide and fragments thereof, including all mutants and variants having full-length or truncated TrkB isoform biological activity as defined here, are included in the methods of the invention. Full-length or truncated TrkB isoform polypeptides can be easily generated using methods well known in the art described, for example, in Sambrook et al. Molecular Cloning: A Laboratory Manual, Cold Spring Harbor Laboratory, New York (1989) and in Ausubel et al, Current Protocols in Molecular Biology, John Wiley & Sons, New York (1997).

In the methods of the invention, full-length or truncated TrkC isoforms may be delivered to neuronal cells in the form of a nucleic acids encoding full-length or truncated TrkC isoforms, preferably using vectors or liposomes, or it may be delivered to cells in the form of a polypeptide, or a mutant, variant, homolog, or fragment thereof having the activity of full-length or truncated TrkC isoforms using liposomes. Thus, the use of full-length or truncated TrkC isoform polypeptide and fragments thereof, including all mutants and variants having full-length or truncated TrkC isoform biological activity as defined here, are included in the methods of the invention. Full-length or truncated TrkC isoform polypeptides can be easily generated using methods well known in the art described, for example, in Sambrook et al. (supra) and in Ausubel et al (supra).

Analogs

The present invention also provides for a method of inhibiting apoptosis using analogs of proteins or peptides encoded by full-length trkB or full length trkC. Analogs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. Conservative amino acid substitutions typically include substitutions within the following groups:

glycine, alanine;

valine, isoleucine, leucine;

aspartic acid, glutamic acid;

asparagine, glutamine;

serine, threonine;

lysine, arginine;

phenylalanine, tyrosine.

Modifications (which do not normally alter primary sequence) include in vivo, or in vitro chemical derivatization of polypeptides, e.g., acetylation, or carboxylation. The invention should be construed to include administration of modified full-length TrkB peptides or full-length TrkC peptides including, but not limited to, peptides modified by glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced is a method of inhibiting apoptosis comprising administration of full-length TrkB peptides or full-length TrkC peptides which have phosphorylated amino acid residues, e.g., phosphotyrosine, phosphoserine, or phosphothreonine.

The invention further includes a method of inhibiting apoptosis by administering full-length TrkB polypeptides or full-length TrkC polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Pharmaceutical Compositions

Pharmaceutical compositions comprising the desired polynucleotide sequences, vectors comprising the same, or peptides encoded thereby, may be formulated and administered to a mammal for inhibition of apoptosis. Such compositions are now described.

The invention encompasses the preparation and use of pharmaceutical compositions comprising a TrkB and/or TrkC compound useful for inhibition of apoptosis as an active ingredient. The invention also encompasses the preparation and use of pharmaceutical compositions comprising polynucleotides encoding anti-sense RNA and/or double-stranded RNA specific for one or more isoforms of truncated TrkB and/or truncated TrkC. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, and other mammals.

Pharmaceutical compositions that are useful in the methods of the invention may be prepared, packaged, or sold in formulations suitable for parenteral, topical, pulmonary, intranasal, buccal, ophthalmic, intrathecal, intracranial, or another route of administration.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intracranial injections, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers. The formulations described herein as being useful for pulmonary delivery are also useful for intranasal delivery of a pharmaceutical composition of the invention.

Typically dosages of the compound of the invention which may be administered to an animal, preferably a human, range in amount from 1 microgram to about 100 grams for proteins and peptides, 10³ to 10⁸ plaque forming units for viruses, and 1 to 500 micrograms for nucleic acids.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

For example, treatment of AD, a chronic disease, may be performed as follows. A viral vector containing polynucleotides encoding anti-sense RNA specific for one or more human truncated TrkB isoforms (SEQ ID NO: 19 and SEQ ID NO: 20) can be given by intranasal spraying, a non-invasive and widely accepted delivery route, although other routes of administration are possible, such as ocular drops. As stated above, 10³ to 10⁸ plaque forming units of the viral vector can be used for infection. Assuming that gene expression does not last more than 20 days, monthly re-exposure will be needed (or at least 10 exposures per year).

To treat an acute disease, the viral vector containing polynucleotides encoding anti-sense RNA specific for one or more human truncated TrkB isoforms (SEQ ID NO: 19 and SEQ ID NO: 20) can be administered as described above. Again assuming that gene expression does not last more than 20 days, re-exposure will only be needed 2 or 3 additional times (4 exposures total).

Examples of acute diseases that could be treated with TrkB and/or TrkC (either full-length, anti-sense RNA, and/or double-stranded RNA specific for a truncated isoform) include stroke, cerebral ischemia, brain trauma, and spinal cord injury. Patients suffering any of these injuries experience neuronal apoptosis and may be treated effectively with TrkB and/or TrkC. These types of injuries require treatment within days of the injury and are excellent candidates for the anti-apoptotic use of TrkB and/or TrkC. Thus, administration of TrkB and/or TrkC is useful in inhibiting apoptosis in both the central nervous system as well as the peripheral nervous system, where it will be particularly effective in cases of spinal cord injury and diabetic neuropathy.

Experiment Methods

For the experiments that are described in detail below, the following methods and reagents are used.

Mouse monoclonal antibody to an extracellular epitope on TrkB [anti-TrkB(out)], which recognizes both full-length TrkB (TrkB.FL) and truncated TrkB (TrkB.T1), was obtained from BD Transduction Laboratories (Lexington, Ky.). Antibodies to the neuron-specific microtubule-associated protein, MAP2ab, and hemagluttin (HA) were obtained from Sigma Chemical Co. (St. Louis, Mo.), and anti-p75 was from Chemicon International Corp (Temecula, Calif.). Rabbit polyclonal antibodies to an intracellular epitope on trkB.FL [TrkB (in)] and to an extracellular epitope on TrkC were provided by Dr. L. Reichardt, UCSF (San Francisco, Calif.). Rabbit polyclonal antibody to an intracellular epitope on the T1 isoform of truncated TrkB [TrkB(T1)] (Yan et al., Immunocytochemical localization of TrkB in the central nervous system of the adult rat, J. Comp. Neurol. 378:135-157 (1997)) was obtained from Dr. S. C. Feinstein, UCSB (Santa Barbara, Calif.). Polyclonal antibody specific for phospho-trk was obtained from New England BioLabs (Beverly, Mass.). Appropriate rhodamine-, fluorescein- or peroxidase-conjugated secondary antibodies were obtained from Jackson ImmunoResearch Laboratories Inc. (West Grove, Pa.). BDNF and NT-3 were supplied by Regeneron Pharmaceuticals (Tarrytown, N.Y.); FGF-2 (basic fibroblast growth factor) was obtained from Upstate Biotechnology Inc. (Lake Placid, N.Y.). TrkB-IgG (provided by Regeneron) is a soluble fusion protein consisting of the extracellular, BDNF binding domain of rat trkB coupled to an Fc fragment of human IgG (Croll et al., Co-infusion with a TrkB-Fc receptor body carrier enhances BDNF distribution in the adult rat brain, Exp. Neurol. 152:20-33 (1998)), which decreases the free extracellular BDNF concentration and inhibits its effects. TrkA-IgG (Regeneron) had no effect on euploid neuron survival demonstrating that there were no non-specific effects of TrkB-IgG (hippocampal neurons do not respond to NGF [Ip et al., Cultured hippocampal neurons show responses to BDNF, NT-3, and NT-4, but not NGF, J. Neurosci. 13:3394-3405 (1993)]).

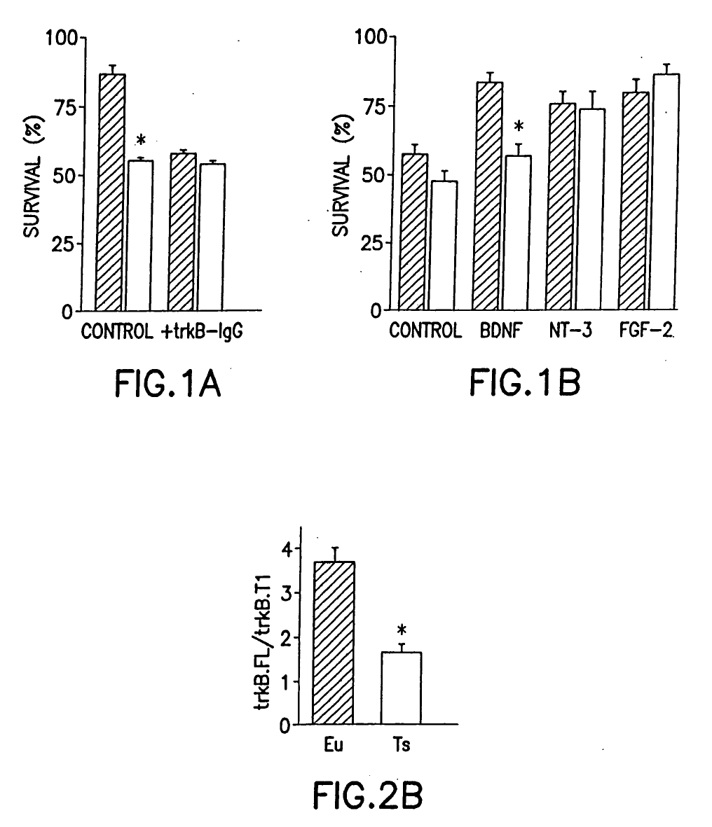

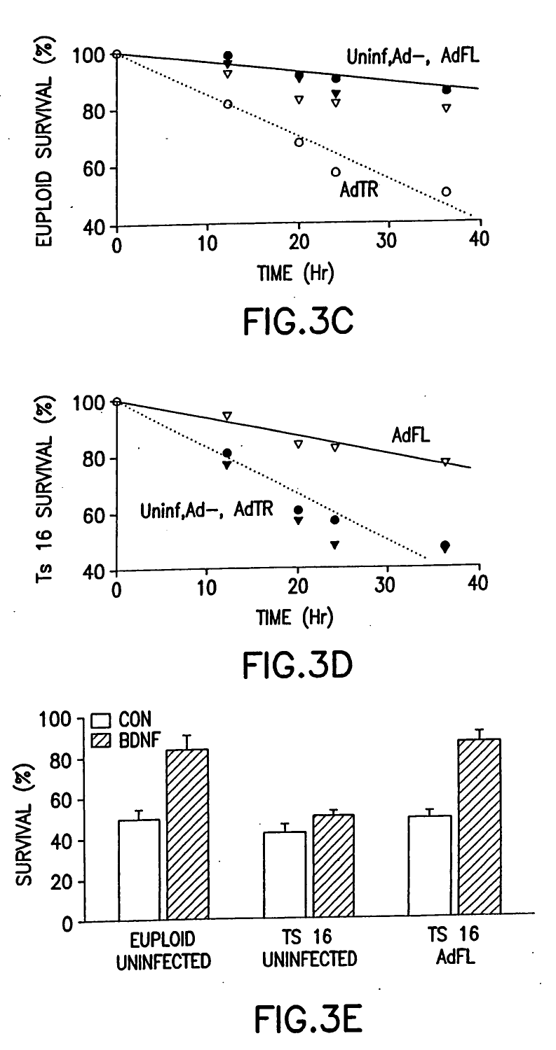

Preparation and characterization of neuron cultures. Hippocampal neurons were cultured from euploid and Ts16 littermate fetuses on embryonic day 15.5 in minimal essential medium (MEM) supplemented with B27 as described in Bambrick et al., Glutamate as a hippocampal neuron survival factor: an inherited defect in the trisomy 16 mouse, Proc. Natl. Acad. Sci. USA 92:9692-9696 (1995). In brief, hippocampi are freed of meninges, digested with trypsin, and dissociated by trituration in MEM 10/10 [MEM with Earle's salts/2 nM glutamine/10% (vol/vol) fetal bovine serum/10% (vol/vol) horse serum/penicillin (100 -units/ml)/streptomycin (100 units/ml)]. Cells are plated in 50,000 cells per cm² on 12-mm glass coverslips photoeteched with a lettered grid of 175 mm×175 mm squares (Eppendorf AG, Hamburg, Germany). The coverslips are pretreated with poly(L-lysine) (Sigma). At 1 day in vitro, the MEM 10/10 is replaced with MEM supplemented with B27. The B27 supplement contains optimized concentrations of neuron survival factors including triiodothyronine, cortisol, transferrin, glutathione, DL-a-tocopherol, and insulin. At 2 days in vitro, the medium is changed to MEM with B27. The cultures are maintained at 37° C. in 95% air/5% CO₂. Each coverslip is kept in a separate well; two to four coverslips are used for each condition in each experiment. Neurons are plated at 10⁴ cells per cm² on 12 mm glass coverslips etched with a lettered grid (Eppendorf AG, Hamburg, Germany) for survival experiments and at 5×10⁵ cells per 35 mm dish for western blots. Initially, (FIG. 1B) coverslips and dishes are coated with poly L-lysine (Sigma); but are changed to coatings of poly L-lysine (Sigma) and merosin (FIG. 1A, and FIGS. 3C-E) because neurons died about half as fast on merosin/poly L-lysine substrate as compared to poly L-lysine alone, however the relative differences between euploid and Ts16 neuron survival and the effects of neurotrophins are identical on the two substrates. Unless otherwise indicated, cell culture reagents are obtained from GIBCO/BRL (Rockville, Md.).

Measurement of neuron survival. At 3 days in vitro, all live neurons in each of five randomly selected, 175 mm×175 mm fields per coverslip (identified by the etched grid) and at least two coverslips per condition were counted using phase contrast microscopy. Cells that had assumed a globular, pyknotic appearance were scored as dead. Separate studies have confirmed that cells scored as live by phase contrast microscopy exclude trypan blue and are not undergoing DNA fragmentation (TUNEL-negative). Depending on the experiment, survival is expressed as the percentage of cells present at 3 days in vitro that remained at 5.5 days in vitro; or, when B27 was removed at 3 days in vitro and the cultures were treated with neurotrophins or FGF-2, survival is expressed as the percentage of neurons present at the time of B27 withdrawal that remained at the end of the treatment period. The significance of differences between euploid and Ts16 cell counts for each condition was determined by student's t-test.

Western blot analysis. SDS-solubilized cell extracts were incubated at 100° C. for five minutes, fractionated on 4-12% NuPAGE bis-tris gels (Invitrogen Corp., Carlsbad, Calif.) and transferred to a nitrocellulose membrane. After blocking in non-fat dried milk, membranes were incubated for 2-16 hours with primary antibody followed by incubation with appropriate peroxidase-conjugated secondary antibodies and visualized by chemiluminescence (ECL, Amersham Pharmacia Biotech Co., Piscataway, N.J.). Blots were quantified by scanning autoradiographs into NIH Image (v 1.62, NIH) to determine the optical density of each band.

Fluorescence immunocytochemistry (ICC). Cultures were fixed in 4% paraformaldehyde and incubated overnight with primary antibody at 4° C. Incubation with rhodamine- or fluorescein-conjugated secondary antibody was for 1 hour. Fluorescence images were acquired using a conventional microscope equipped with epifluorescence optics (Olympus America Co., Melville, N.Y.) or a confocal microscope (Model LSM410; Carl Zeiss, Jena, Germany).

Replication-deficient recombinant adenoviruses. Adenoviruses were generated as described in Gonzalez et al., Disruption of TrkB-mediated signaling induces disassembly of postsynaptic receptor clusters at neuromuscular junctions, Neuron 24:567-583 (1999). In brief, the pAdLink plasmid, containing the cytomegalovirus (CMV) promoter/enhancer, an SV40 polyadenylation sequence, and flanking adenovirus backbone sequences, was modified by inserting multiple cloning sites, an IRES from pLIGns, and green fluorescent protein (GFP) (codon-corrected cDNA; GIBCO-BRL). cDNAs encoding other transgenes were then cloned into this plasmid. Recombinant, replication-defective adenovirus was generated by homologous recombination with the viral Ad5, E1a-deleted d1327 backbone in human embryonic kidney 293 stem cells that are permissive for viral replication. The Escherichia coli lacZ gene encoding b-gal and the gene for GFP were cloned into pAdLink, and adenovirus was generated. Ad- encodes lacZ and GFP under control of the CMV promoter and an IRES sequence and serves as a control for nonspecific effects of viral infection and over-expression of exogenous protein. A mouse truncated TrkB.T1 cDNA and mouse full-length TrkB cDNA (TrkB.FL) were epitope tagged at the carboxyl terminus of the protein with hemagluttinin (HA) and these genes and the gene for GFP were cloned into the modified pAdLink plasmid. Purified virus was generated after three rounds of plaque selection by a limiting dilution method in 293 cells. The integrity of the viral genome was examined by Southern blot, and the absence of wild-type Ad5 virus was confirmed by PCR using primers specific to the deleted E1a region. Virus was resuspended in HEPES-buffered saline (HBS [pH 7.8]) 10% glycerol, particle density was measured spectrophotometrically at OD₂₆₀, and pfu was determined by plaque assays on agar overlays using a limiting dilution method. Virus aliquots of 1×10¹² pfu/ml were stored at −70° C. for <4 months, and viral stocks were stored in liquid N₂. The hemagglutinin (HA) sequences at the C-terminus of the TrkB.FL and TrkB.T1 enable detection of the exogenous TrkB proteins, independently of endogenous TrkB proteins. In these vectors, GFP was under the control of the CMV promoter and an IRES sequence to allow translation of a bicistronic message. The adenovirus designated AdTR contains DNA which encodes the mouse truncated TrkB isoform (TrkB.T1) (cDNA gift of Dr. M. Barbacid) (SEQ ID NO: 11). It is noted that AdTR lacks the intracellular tyrosine kinase domain of TrkB. The adenovirus designated AdFL contains DNA which encodes the mouse full-length TrkB (TrkB.FL) (SEQ ID NO: 9). Anti-HA immunostaining is used as an indicator of AdFL and AdTR infection in this study; GFP fluorescence is used to confirm infection by Ad- (75% of neurons were infected). Adenovirus mediated transgene expression and function are evaluated by western blot, ICC, and in a PC12 neurite outgrowth assay as described in Gonzalez et al., (supra). An in vitro assay was used to determine whether virally expressed trkB.T1 could decrease BDNF or NT4/5 signaling through endogenous, full-length TrkB in a dominant-negative fashion. A stably transfected PC12 cell line that expresses TrkB.FL (PC12-trkB) was used; these cells extend neurites in the presence of BDNF. Cells were plated at low-passage number and maintained in medium with 10% horse serum, 5% fetal bovine serum, penicillin (100 units)/streptomycin (100 mg) at 37° C. in 5% CO₂. One day after splitting, cells were infected with AdTR or Ad- (2×10⁸ pfu/10⁴ cells), or vehicle. Three days later, 1-100 ng/ml BDNF, NT-4/5 or NGF was added to the medium for 5 days. Cells that were treated with AdTR did not extend neurites in response to BDNF whereas Ad- or untreated cells produced extensive neurites in response to BDNF. As a positive control to evaluate nonspecific effects of viral infection, neurite extension was examined in another cell line (PC63) which expresses TrkA. These cells were also infected with AdTR and Ad-. Neither virus prevented the ability of NGF to stimulate neurite growth in these cells.

### Accelerated Death of Ts16 Neurons Due to Failure of BDNF Signaling

Cultures of normal (euploid) and Ts16 neurons were prepared from embryonic littermate hippocampi and maintained in serum-free medium (MEM) containing the chemically-defined supplement, B27 (Brewer et al., Optimized survival of hippocampal neurons in B27-supplemented Neurobasal, a new serum-free medium combination, J. Neurosci. Res. 35:567-576 (1993)). The cultures contained almost exclusively postmitotic neurons.

Both euploid and Ts 16 cultures contained >95% MAP2ab-immunoreactive neurons with the remainder being flat cells identified as astrocytes by GFAP ICC. The proportion of glial cells was the same in euploid and Ts16 cultures.

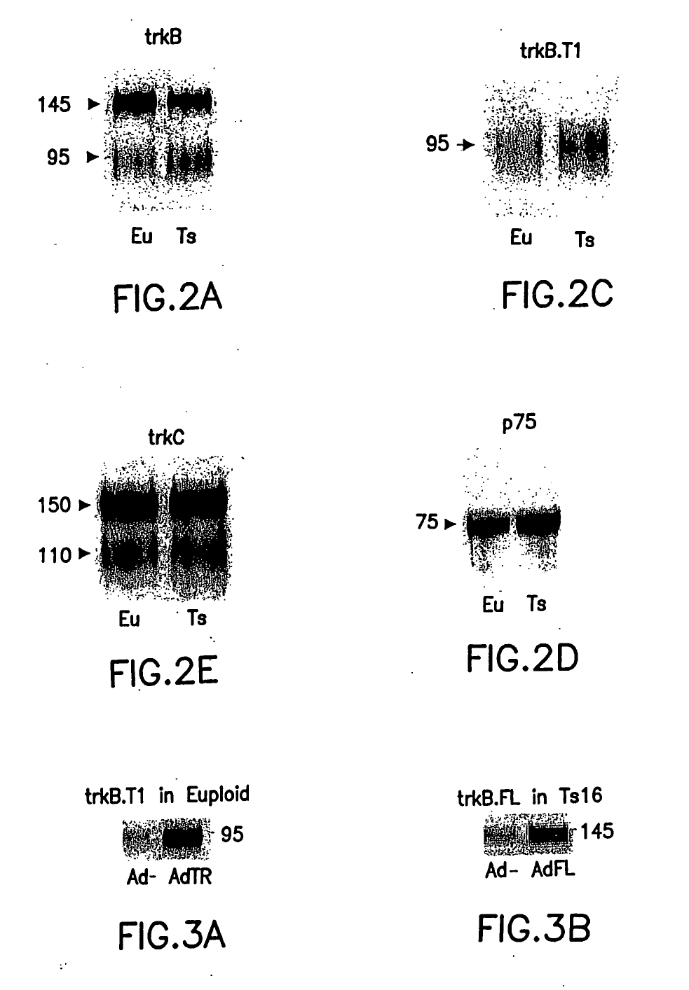

Cortical astrocytes, cultured from euploid and Ts16 littermate fetuses as previously described (Bambrick L L, et al., Expression of glial antigens in mouse astrocytes: species differences and regulation in vitro, J. Neurosci. Res. 46:305-15 (1996)), contained the same amount of TrkB.T1 by western blot analysis, demonstrating that differences in TrkB.T1 expression (FIGS. 2A, 2B, and 2C) were not due to differences in TrkB.T1 levels in contaminating astrocytes.

By 3 days in vitro, neurons from both genotypes took on the characteristics of differentiated neurons with extensive processes. At this time there were no differences in soma size or in neurite length or branching between the two genotypes. Some cells in both euploid and Ts16 cultures died over 5 days in vitro. Ts16 neurons die about three-times faster than euploid neurons (Bambrick et al., supra (1995); Bambrick and Krueger, Neuronal apoptosis in mouse trisomy 16: mediation by caspases, J. Neurochem. 72:1769-1772 (1999)). Similarly, in the present study, about 13% of euploid and about 42% of Ts16 neurons died over a 2.5-day period (FIG. 1A). Addition of TrkB-IgG (2 mg/ml) at 3 days in vitro (Croll et al., supra (1998)) to deplete endogenous BDNF from the medium reduced the survival of euploid neurons to Ts16 levels without affecting Ts16 neuron survival (FIG. 1A). Survival is expressed as % of cells present at 3 days in vitro that were still present at 5.5 days in vitro. This lack of survival demonstrates that BDNF is normally secreted in euploid hippocampal neuron cultures where it promotes neuron survival and that this autocrine BDNF-mediated survival pathway is not functioning in Ts16 cultures.

In order to determine whether Ts16 neurons were capable of responding to BDNF, B27 was removed at 3 days in vitro and the ability of exogenous BDNF alone to support neuron survival was determined. Removal of B27 caused about half of both euploid and Ts16 neurons to die within one day. In euploid neurons, this death was blocked by BDNF (100 ng/ml) addition at 3 days in vitro (after B27 removal), whereas the Ts16 neurons were not rescued by the exogenous BDNF (FIG. 1B). Survival is expressed as % of cells present at 3 days in vitro that were still present at 4.5 days in vitro. In MEM+BDNF, 16% of euploid neurons and 50% of Ts16 neurons died. Error bars show sem (n=3) and * indicates euploid and Ts16 survival were significantly different by t-test (p<0.001). BDNF failed to rescue Ts16 neurons even at 1 mg/ml, ten times the maximally-effective concentration for euploid neurons.

TrkA-IgG had no effect on euploid neuron survival demonstrating that there were no non-specific effects of TrkB-IgG [mouse hippocampal neurons do not respond to NGF (N.Y. Ip, et al, supra (1993))].

To determine whether Ts16 neurons are capable of responding to other survival factors, B27 was withdrawn at 3 days in vitro and replaced with BDNF (100 ng/ml), NT-3 (100 ng/ml), or basic fibroblast growth factor (FGF-2) (10 ng/ml). Survival is determined as % of cells present at the time of B27 withdrawal that were still alive 16 hours later. Survival of euploid neurons in the presence of BDNF, NT-3, and FGF-2 was significantly different (p<0.05) from that in the absence of survival factors (vehicle). Survival of Ts16 neurons in the presence of NT-3 and FGF-2, but not in the presence of BDNF, was significantly different (p<0.05) from that in the absence of survival factors. Even though BDNF was unable to promote the survival of Ts16 neurons, NT-3 and FGF-2 rescued both euploid and Ts16 neurons to the same extent. Thus, Ts16 neurons have a selective failure of the survival response to BDNF.

### Ts16 Neurons Overexpress Truncated trkB

In order to determine whether Ts16 neurons lack the BDNF receptor, TrkB, the TrkB composition of euploid and Ts16 cultures was analyzed by western blotting with an antibody [anti-TrkB(out)] that recognizes the extracellular domain of the receptor (FIG. 2A). FIG. 2A shows the western blot of euploid and Ts16 hippocampal neurons using anti-TrkB(out), which binds to a common epitope on the extracellular side of full length (145 kDa) and truncated (95 kDa) TrkB. The western blot was performed as described above. Rabbit polyclonal antibodies to an intracellular epitope on TrkB.FL [TrkB(in)] and to an extracellular epitope on TrkC were used as well as rabbit polyclonal antibody to an intracellular epitope on TrkB.T1.

In FIG. 2A, euploid and Ts16 neurons expressed both the full-length, functionally active isoform, TrkB.FL (145 kDa) (full-length TrkB) and the catalytically inactive, truncated isoform, TrkB.T1 (95 kDa) (truncated TrkB), which has been proposed to inhibit BDNF signaling via TrkB by a dominant-negative mechanism (Middlemas et al., supra (1991); Eide et al., supra (1996)). Although Ts16 neurons expressed slightly less TrkB.FL, they expressed substantially more TrkB.T1. The ratio of TrkB.FL to TrkB.T1 expresssion was 3.8 in euploid neurons and only 1.5 in Ts16 neurons (see FIG. 2B where the error bars show sem (n=3; *, p<0.05)). Overexpression of TrkB.T1 was confirmed using an antibody (Fryer R H, et al., Developmental and mature expression of full-length and truncated trkB receptors in the rat forebrain, J. Comp. Neurol. 374:21-40 (1996)) to the unique, intracellular domain of the T1 isoform of TrkB.T1 (see FIG. 2C in which anti-TrkB(T1) was used to label an internal epitope on TrkB.T1). The neurotrophins also bind to the low-affinity neurotrophin receptor, p75, which may modulate neurotrophin-mediated neuron survival in the absence of trk receptors (Casaccia-Bonnefil, P, et al., Neurotrophins: the biological paradox of survival factors eliciting apoptosis, Cell Death Differ. 5:357-364 (1998)), however, p75 expression was the same in euploid and Ts16 neurons (FIG. 2D). In addition, the expression of the NT-3 receptor, TrkC, and its truncated isoforms was the same in euploid and Ts16 neurons (FIG. 2E which shows a western blot of euploid and Ts16 neurons using an antibody to TrkC that labels both full length (150 kDa) and truncated (110 kDa) isoforms), consistent with the survival-promoting effect of NT-3 in both genotypes (FIG. 1C).

In order to rule out the possibility that Ts16 cultures contain a higher proportion of neurons that express only TrkB.T1, euploid and Ts16 cultures were analyzed by fluorescence immunocytochemistry (ICC) using anti-TrkB(T1) and anti-TrkB(in), which recognizes a unique, intracellular epitotope of the full-length TrkB isoform. All of the neurons in both euploid and Ts16 cultures expressed both TrkB.FL and TrkB.T1. The cellular distributions of the two isoforms were similar, with expression present in the plasma membrane and cytoplasm; the distributions were indistinguishable in the two genotypes. This intracellular distribution is consistent with reports that TrkB is present in both plasma membrane and intracellular locations and can be redistributed in response to physiological stimuli (Meyer-Franke A, et al., Depolarization and cAMP elevation rapidly recruit TrkB to the plasma membrane of CNS neurons, Neuron 21:681-693 (1998); Du J, et al., Activity- and Ca²⁺-dependent modulation of surface expression of brain-derived neurotrophic factor receptors in hippocampal neurons, J. Cell. Biol. 150:1423-1433 (2000)).

### BDNF-Stimulated TrkB Phosphorylation is Reduced in Ts16 Neurons

If TrkB.T1 acts by a dominant negative mechanism to reduce TrkB signaling, there should be less BDNF-stimulated tyrosine phosphorylation of TrkB in Ts16 neurons. To test this prediction phosphorylation of TrkB was measured by western blot analysis using antibodies specific for phosphotyrosine in position Y490 in TrkB.FL. This antibody was raised to phospho-TrkA and it also recognizes the corresponding phosphorylated tyrosine in TrkB and TrkC. Because there is no detectable TrkA in mouse hippocampal neurons and any BDNF-stimulated phospho-TrkC could be distinguished on the basis of molecular size on these gels, in mouse hippocampal neurons, the BDNF-induced increase in trk phosphorylation determined with this antibody is phospho-TrkB. Euploid and Ts16 neuron cultures were preincubated without B27 for 4 hours and then in the absence or presence of 100 ng/ml BDNF for 5 minutes. Cells were subjected to western blot analysis as described above using anti-phospho-Trk (P-TrkB) or TrkB(out) (TrkB).

There was no detectable phosphorylation of TrkB in the absence of BDNF while 100 ng/ml BDNF caused a dramatic increase in TrkB phosphorylation. There was about 33% less TrkB phosphorylation in Ts16 neurons. The predicted change in BDNF/TrkB signaling via full-length homodimers for any reduction in the TrkB.FL/TrkB.T1 ratio can be computed assuming a dominant negative mechanism of inhibition by the truncated isoform (Eide et al., supra (1996)). Based on the observation that the TrkB.FL/TrkB.T1 ratio is 3.8 in euploid neurons and 1.5 in Ts16 neurons, this calculation predicts a 37% decrease in full-length TrkB homodimers and, therefore, in BDNF-stimulated TrkB autophosphorylation in the Ts16 neurons (p<0.05, n=4). Thus, BDNF stimulation of TrkB tyrosine phosphorylation is reduced in Ts16 neurons by an amount predicted from the measured decrease in the TrkB.FL/TrkB.T1 ratio.

### Expression of Exogenous TrkB.FL in Ts16 Neurons Restores BDNF Survival Signaling

Overexpression of TrkB.T1 relative to TrkB.FL could cause the failure of BDNF signaling in Ts16 neurons. In order test this hypothesis, replication-deficient adenoviruses were utilized to introduce TrkB.FL or TrkB.T1 into the neurons in order to experimentally manipulate the proportions of the two trkB isoforms. The replication-deficient adenoviruses contained DNA coding for TrkB.FL (SEQ ID NO: 9) (AdFL), TrkB.T1 (SEQ ID NO: 11) (AdTR), or no TrkB DNA (Ad-) and were generated as described above (see also Gonzalez M, supra (1999)).

Euploid and Ts16 neurons infected with AdTR expressed increased levels of TrkB.T1 as detected by either anti-TrkB(out) or anti-TrkB(T1) (TrkB.T1 in euploid neurons illustrated in FIG. 3A). In FIG. 3A, euploid neurons were exposed to adenovirus carrying TrkB.T1-HA DNA (AdTR) resulting in expression of TrkB.T1 detected on western blots, at 95 kDa, using anti-TrkB(out). Anti-HA ICC revealed that the exogenous TrkB.T1 was expressed in the plasma membrane and cytoplasm. Similarly, euploid and Ts16 neurons infected with AdFL expressed increased amounts of TrkB.FL (TrkB.FL in Ts16 neurons illustrated in FIG. 3B). In FIG. 3B, Ts16 neurons were exposed to adenovirus carrying TrkB.FL-HA DNA (AdFL) resulting in expression of TrkB.FL detected on western blots using anti-TrkB(out). Anti-HA ICC revealed that like exogenous TrkB.T1, exogenous TrkB.FL was expressed in the plasma membrane and cytoplasm. ICC using anti-HA revealed that 75% of the neurons expressed exogenous TrkB.T1 or TrkB.FL, moreover, examination of expression of the HA tag by fluorescence confocal ICC revealed that most of the exogenous TrkB.T1 and TrkB.FL in infected neurons was located on the plasma membrane. Ad- did not affect levels or distribution of endogenous TrkB.FL and TrkB.T1.

Neuron survival was studied in cultures infected with Ad-, AdFL and AdTR (FIGS. 3C, D, E). Time courses of neuron survival in the presence of BDNF are shown for euploid (FIG. 3C) and Ts16 (FIG. 3D) neurons. Ad- and AdFL did not substantially affect the BDNF-induced survival of euploid neurons. In contrast, AdTR, which raised TrkB.T1 expression (FIG. 3A), increased the rate of euploid neuron death (FIG. 3C, dotted line) to a level approximately equal to the rate of death of uninfected Ts16 neurons in the presence of BDNF (100 ng/ml). In FIG. 3C, expression of TrkB.T1 in euploid neurons inhibited BDNF survival signaling. Euploid neurons were either left untreated (·,Uninf) or treated with Ad- (t), AdFL (Ñ) or AdTR (O) at 2 days in vitro. At 3 days in vitro, B27 was withdrawn from the cultures and 100 ng/ml BDNF was added. Surviving neurons were repeatedly counted in 5 identified fields on each of two coverslips per condition. 250-400 neurons were counted for each data point. In FIG. 3C, the solid line represents a linear regression for data for the untreated neurons, and the dotted line represents a linear regression for AdTR-treated neurons.

When added to Ts16 cultures (FIG. 3D), AdTR slightly increased the rate of neuron death while Ad- had no effect. In contrast, AdFL increased Ts16 neuron survival in the presence of BDNF to the level of survival of euploid neurons in the presence of BDNF (FIG. 3D, dotted line). In FIG. 3D, the expression of TrkB.FL in Ts16 neurons restored BDNF survival signaling. Ts16 neurons were either untreated (·, Uninf) or treated with Ad- (t) AdTR (O) or AdFL (Ñ) at 2 days in vitro. At 3 days in vitro, B27 was withdrawn from the cultures and 100 ng/ml BDNF was added. Surviving neurons were repeatedly counted in 5 identified fields on each of two coverslips under each condition. 250-400 neurons were counted for each data point. In FIG. 3D, the solid line represents a linear regression for data for the untreated neurons, and the dotted line represents a linear regression for AdFL-treated neurons.

The essential findings of the effect of TrkB.FL expression on BDNF survival signaling are summarized in FIG. 3E. Data show mean±sem (n=3 experiments) survival 36 hours after B27 withdrawal. About half of the untreated euploid neurons died in the absence of 100 ng/ml BDNF while fewer than 20% died in its presence. BDNF did not increase survival of untreated Ts16 neurons, however, in Ts16 neurons treated with AdFL, BDNF elicited a survival response that was indistinguishable from that of euploid neurons. BDNF reverses approximately 65% of the euploid neuron death induced by B27 withdrawal but has no effect on Ts16 neuron survival. Infection of Ts16 neurons with AdFL, which raises expression of TrkB.FL (FIG. 3B), completely restores the ability of BDNF to rescue the Ts16 neurons. In addition, raising TrkB.FL in Ts16 neurons also prevents the appearance of fragmented neurites, a characteristic of early stages of neuronal apoptosis. Cultured neurons were incubated in the absence of B27 and the presence of 100 ng/ml BDNF for 36 hours and then immunostained for MAP2ab using a rhodamine-conjugated secondary antibody. Most euploid neurons had smooth neurites. In contrast, many surviving Ts16 neurons had fragmented neurites indicative of early neurodegeneration. Ts16 neurons treated with AdFL had very few fragmented neurites and the cultures were morphologically indistinguishable from euploid neurons.

These results demonstrate that a chromosomal abnormality in mice (Ts16) with considerable similarity to DS (Ts21) results in the selective failure of BDNF-induced survival signaling. Not wishing to be bound by theory, this failure appears to be result from the elevated expression of a truncated isoform of the BDNF receptor, TrkB. Without excluding a role for signaling by TrkB.T1 (Haapasalo A, et al., Expression of the naturally occurring truncated trkB neurotrophin receptor induces outgrowth of filopodia and processes in neuroblastoma cells, Oncogene 18: 1285-1296 (1999), Baxter G T, et al., Signal transduction mediated by the truncated trkb receptor isoforms, trkB.T1 and trkB.T2, J. Neurosci. 17:2683-2690 (1997)), it is clear that elevated expression of TrkB.T1 in Ts16 neurons would reduce BDNF signaling by forming TrkB.T1-TrkB.FL heterodimers that are incapable of signaling to downstream effectors due to the absence of trans-tyrosine auto-phosphorylation (Eide F F, et al., supra (1996); Gonzalez M, et al., supra (1999); Ichinose and Snider, Differential effects of TrkC isoforms on sensory axon outgrowth, J. Neurosci. Res. 59:365-371 (2000); Yacoubian and Lo, Truncated and full-length TrkB receptors regulate distinct modes of dendritic growth, Nature Neurosci. 3:342-349 (2000)). It is of interest that the TrkB.FL/TrkB.T1 ratio in Ts16 neurons (FIG. 2B) predicts only a 37% decrease in trk phosphorylation (Eide F F, et al., supra (1996)). This predicated decrease is consistent with the finding of BDNF-induced TrkB phosphorylation in both euploid and Ts16 neurons, indicating that some of the TrkB.FL in Ts16 neurons does form functionally active homodimers (western blotting with anti-phospho-trk).

It is of interest that TrkB.T1 is elevated in hippocampal and cortical neurons of AD patients (Ferrer I, et al., BDNF and full-length and truncated TrkB expression in Alzheimer disease. Implications in therapeutic strategies, J. Neuropathol. Exp. Neurol. 58:729-739 (1999)). By altering the expression of truncated trkB and full length trkB in AD patients, one may be able to treat AD patients.

BDNF regulates other neural functions including the generation and differentiation of neurons during development, axon growth and growth cone mobility, and synaptic plasticity (Lu supra (1999)). If one or more of these BDNF-mediated responses were affected in DS because of elevated truncated trkB expression, cognitive function could be compromised due to errors in connectivity and the failure to properly modulate synaptic plasticity, even before significant numbers of neurons are lost. Such deficits could contribute to mental retardation and premature AD in this disorder. However, increasing the level of expression of full-length trkB or reducing the amount of truncated TrkB polypeptides in the neurons may prevent some or all of the cognitive function impairment. Improved connectivity and modulation of synaptic plasticity may result from increasing the amount of full-length TrkB expressed in neurons or decreasing the amount of truncated TrkB expressed in neurons.

The importance of neurotrophins in maintaining neuron survival has led to attempts to introduce neurotrophins into the brain in order to treat neuro-degenerative disorders such as AD and Parkinson's disease (Lu, supra (1999)). The results reported here raise the possibility that failure of neurotrophin signaling may contribute to some neuro-degenerative disorders and, consequently, affected neurons may not respond to therapies designed to raise neurotrophin levels in the brain. Finally, the ability to reverse a naturally-occurring failure to respond to a neuron survival factor by introducing a particular isoform of its receptor suggests potential therapeutic strategies for treatment of neuro-degenerative disorders.

### Reduction of TrkB. T1 Levels in Ts16 Neurons

In order to reduce the amount of TrkB.T1 polypeptide in Ts16 neurons, one can express within the neuron or administer to the neuron anti-sense RNA whereby the anti-sense RNA is complementary to a portion of the TrkB.T1 nucleotide sequence that is specific to the truncated isoform. Also, one can express within a neuron or administer to a neuron double-stranded RNA with sequences specific for TrkB.T1. These methods will result in a measurable decrease (by western blot) in the amount of TrkB.T1 isoform present in the neurons.

### A. Adenovirus Mediated Administration

To express anti-sense RNA in Ts16, any of the above mentioned viral vectors can be used to introduce the polynucleotide into the cells. In one example, one can use adenovirus containing 1089 base pair of DNA (SEQ ID NO: 17) which one uses to generate anti-sense RNA. The 1089 base pair anti-sense RNA is complementary to the mRNA for TrkB.T1 in the unique T1 intracellular domain and 3′ UTR regions. The anti-sense RNA for this example is the same as SEQ ID NO: 17 but with uracil instead of thymine. It is possible to use shorter lengths of DNA in the adenovirus to generate shorter anti-sense RNA, so long as the adenovirus generates an anti-sense RNA that is complementary to the mRNA in a region specific for T1. An adenovirus vector containing the anti-sense RNA sequences is generated generally as described above (see also Gonzalez et al., supra (1999)) except that the DNA sequences encodes the anti-sense RNA (SEQ ID NO: 17) for mouse TrkB.T1. No HA and GFP sequences need to be added to the adenovirus. This construct is designated AdTR.anti. Adenovirus mediated transgene expression and function are evaluated by western blot and in a PC12 neurite outgrowth assay as described supra.

Ts16 neurons infected with AdTR.anti have reduced levels of truncated TrkB as determined by western blot (as described above) using either anti-TrkB(out) or anti-TrkB(T1).

Neuron survival is studied in cultures of Ts16 neurons infected with ADTR.anti. Time courses of neuron survival in the presence of BDNF indicate that Ts16 neurons infected with AdTr.anti have better survival compared to Ad-infected Ts16 neurons. For survival studies, Ts16 neurons are infected with AdTr.anti or Ad- at 2 days in vitro. At 3 days in vitro, B27 is withdrawn from the cultures and 100 ng/ml of BDNF is added. Surviving neurons are repeated counted in 5 identical fields on each of two coverslips per condition. 250-400 neurons are counted for each data point. Thus, the reduction in the amount of TrkB.T1 in Ts16 neurons leads to improved survival of the cells.

### B. Addition of Anti-Sense RNA Oligos to Media Administration

Administration of anti-sense RNA can occur via the addition of oligos of RNA (ranging in length from 10 mer to 45 mer, and more preferably from 18 mer to 25 mer) to the cell culture media at a concentration of 0.1 mM to 500 mM, more preferably between 1 mM to 50 mM. The cells in culture are Ts16 neurons, isolated as described above. The anti-sense oligonucleotide administered is specific to the T1 isoform of truncated Trk.B. One possible sequence is AAGCAGGCUG CAGACAUCCU (SEQ ID NO: 18). It is possible to use thymine instead of uracil in the anti-sense RNA. This sequence can be produced using any known in the art nucleotide generators (Oligos Etc., Wilsonville, Oreg.).

One to five days after addition of the anti-sense RNA oligos to the cell culture media which contains B27, the Ts16 cells are harvested and the amount of TrkB.T1 isoform present in the cells is determined via western blot (as described above) using either anti-TrkB(out) or anti-TrkB(T1). The amount of TrkB.T1 isoform in the Ts16 neurons with anti-sense RNA oligos added to the cell culture media decreases compared to untreated Ts16 neurons with no effect on the amount of full-length TrkB.

To test increased survival of Ts16 neurons having anti-sense RNA added to the cell culture media, the Ts16 neurons are kept in culture with between 1 mM to 50 mM anti-sense RNA (SEQ ID NO: 18) for five days. After five days of culture in B27 supplemented media with anti-sense RNA, the B27 and anti-sense RNA are removed and 100 ng/ml of BDNF is added along with anti-sense RNA (1 mM to 50 mM). Surviving neurons are counted daily in 5 identical fields on each of two coverslips per condition. 250-400 neurons are counted for each data point. The addition of anti-sense RNA oligos to the cell culture media increases the survival of the Ts16 neurons compared to the survival of untreated Ts16 neurons.

### C. RNA Interference (RNAi) via Adenovirus Administration

Eukaryotic gene expression can be effectively inhibited by double-stranded RNA molecules. It is generally accepted in the art-field that the double-stranded RNA molecules efficiently inactivate transcribed genes for long periods of time. This process is called RNA interference (RNAi) or RNA silencing. Double-stranded RNA can be introduced into neurons via adenovirus mediated gene therapy, electroporation, micro-injection, or calcium phosphate precipitation, or any of the other methods described above.

Use of replication-defective adenovirus may be particularly useful in this method. Any of the sequences described for anti-sense RNA adenovirus gene therapy or anti-sense RNA oligos can be cloned into replication-defective adenovirus vectors as described above. In addition, another promoter (such as neuron-specific enolase) is cloned into the 3′ end of the DNA sequence such that the promoter is orientated to drive transcription of the negative or complementary DNA strand, thereby allowing generation of two complementary strands of mRNA which can then hybridize and form double-stranded RNA.

### Treatment or Prevention of Neuro-Degenerative Disorders and Neuro-Developmental Disorders

The above experiments indicate that one can increase the survival of Ts16 neurons by either increasing the amount of full-length TrkB or decreasing the amount of truncated TrkB in the neurons. Because Ts16 is a well-known mouse model for Downs Syndrome and because neurons for various human neurogenerative diseases lack an ability to survive even when BDNF, NT-4/5, and NT-3 are administered, it is proposed that altering the level of truncated isoforms of TrkB and/or TrkC in cells may treat or prevent various neuro-degenerative diseases. One can decrease the levels of truncated TrkB and/or TrkC in cells by using anti-sense RNA and/or double-stranded RNA technology and gene therapy. Alternatively, one can increase the levels of full-length TrkB and/or TrkC in cells by using gene therapy. Alternatively, one can both decrease the level of expression of truncated TrkB and/or TrkC while, at the same time, increasing the level of expression of full-length TrkB and/or TrkC.

It is possible to treat neuro-degenerative disorders and neuro-developmental disorders by altering the ratio of the amount of human full-length TrkB (TrkB.FL) polypeptide (SEQ ID NO: 2) to human truncated TrkB isoform TrkB.T1 polypeptide (SEQ ID NO: 4) and/or human truncated TrkB isoform TrkB.Shc (SEQ ID NO: 6) in cells. One can increase this ratio by increasing the amount of full-length TrkB polypeptide and/or decreasing the amount of truncated TrkB polypeptides (either TrkB.T1 or TrkB.Shc or a combination of both). One can decrease this ratio by increasing the amount of truncated TrkB polypeptides (either TrkB.T1 or TrkB.Shc or a combination of both) and/or decreasing the amount of full-length TrkB polypeptide.

One can increase the amount of full-length TrkB protein in neurons by getting DNA into neurons by using any of the methods of administration described above. For example, DNA encoding for human full-length TrkB (SEQ ID NO: 2) can be cloned into a replication-defective adenovirus as described above. Then 10³ to 10⁸ plaque forming units of the adenovirus vector can be administered intra-nasally on a monthly basis.

In the event that one desires to selectively induce apoptosis, then one can take a similar approach as described above but instead increase the amount of truncated TrkB protein (TrkB.T1 and/or TrkB.Shc) expressed in cells. DNA encoding for TrkB.T1 (SEQ ID NO: 4) or TrkB.Shc (SEQ ID NO: 6) is cloned into a replication-defective adenovirus as described above. Then 10³ to 10⁸ plaque forming units of the adenovirus vector can be administered intra-nasally on a monthly basis.

It is possible to decrease the amount of truncated TrkB protein in a cell by using any of the above mentioned vectors or techniques. One would need to utilize the human TrkB.T1 and/or human TrkB.Shc sequences which are described above.

Similarly, if one desires to selectively induce apoptosis, then one can take a similar approach as described above using double-stranded RNA or anti-sense RNA specific for full-length TrkB or TrkC to decrease the amount of full-length TrkB protein or full-length TrkC protein in cells.

In addition to altering the ratio of the amount of full-length TrkB protein to the amount of truncated TrkB proteins in cells or the ratio of the amount of full-length TrkC protein to the amount of truncated TrkC proteins in cells, one may also administer growth factors (such as BDNF, NT-3, NT-4/5, B27, or other neurotrophins) or antagonists or agonists which bind to the TrkB receptor or TrkC receptor to help in the treatment and/or prevention of the neuro-degenerative or neuro-developmental disorders or other diseases.

It is also understood that TrkB and TrkC are expressed in various tissues in addition to neuronal tissue. Diseases which adversely affect these tissues can be treated in a similar manner as described above by altering the ratio of the amount of the isoform proteins present in those cells. Application of growth factors, other proteins, antagonists, and/or agonists which bind to the TrkB and/or TrkC receptors is useful to treat or prevent the diseases.

It is appreciated that details of the foregoing embodiments, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

1. A method of treating a neuro-degenerative disorder or a neuro-developmental disorder, said method comprising:
contacting a neuropathic hippocampal neuron with an amount of an isolated nucleic acid encoding full-length TrkB or any mutant, variant, homolog, or fragment thereof having the same activity as said full-length TrkB, whereby said amount of said isolated nucleic acid is sufficient to increase the amount of full-length TrkB in said neuron compared to an untreated neuron.

2. The method of claim 1, wherein said nucleic acid sequence encodes the amino acid sequence of SEQ ID NO:2.

3. The method of claim 1, wherein said nucleic acid comprises the nucleotide sequence of SEQ ID NO:1.

4.-12. (canceled)

13. A method of a neuro-degenerative disorder or a neuro-developmental disorder, said method comprising
contacting a neuropathic hippocampal neuron with an amount of a vector sufficient to alter the ratio of amount of full-length TrkB polypeptide to truncated TrkB polypeptide in said neuron.

14. The method of claim 13, wherein said vector comprises a nucleic acid encoding for full-length TrkB.

15. The method of claim 13, wherein said vector is selected from the group consisting of a virus and a plasmid.

16. The method of claim 15, wherein said virus is selected from the group consisting of a herpes virus, adenovirus, adeno associated virus, retrovirus, vacccinia virus, and canary pox virus.

17. (canceled)

18. The method of claim 14, wherein said nucleic acid comprises a nucleotide sequence encoding the amino acid sequence of SEQ ID NO:2.

19.-54. (canceled)


SEQUENCE LISTING (22 sequences)

SEQ ID NO: 1 - DNA, 3707 bp, Homo sapiens
  ref <308>: DATABASE ACCESSION NUMBER: NCBI/NM_006180
  ref <309>: DATABASE ENTRY DATE: 2000-11-01
  ref <313>: RELEVANT RESIDUES: (1)..(3707)
cccccattcgcatctaacaaggaatctgcgccccagagagtcccggacgccgccggtcggtgcccggcgcgccgggccatgcagcgacggccgccgcggagctccgagcagcggtagcgcccccctgtaaagcggttcgctatgccgggaccactgtgaaccctgccgcctgccggaacactcttcgctccggaccagctcagcctctgataagctggactcggcacgcccgcaacaagcaccgaggagttaagagagccgcaagcgcagggaaggcctccccgcacgggtgggggaaagcggccggtgcagcgcggggacaggcactcgggctggcactggctgctagggatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggctagggcccttttccccagaccgatccttcccaacgtactcctcagacgggctgagaggatgaacatcttttaactgccgctggaggccaccaagctgctctccttcactctgacagtattaacatcaaagactccgagaagctctcgagggaagcagtgtgtacttcttcatccatagacacagtattgacttctttttggcattatctctttctctctttccatctcccttggttgttcctttttctttttttaaattttctttttcttcttttttttcgtcttccctgcttcacgattcttaccctttcttttgaatcaatctggcttctgcattactattaactctgcatagacaaaggccttaacaaacgtaatttgttatatcagcagacactccagtttgcccaccacaactaacaatgccttgttgtattcctgcctttgatgtggatgaaaaaaagggaaaacaaatatttcacttaaactttgtcacttctgctgtacagatatcgagagtttctatggattcacttctatttatttattattattactgttcttattgtttttggatggcttaagcctgtgtataaaaaagaaaacttgtgttcaatctgtgaagcctttatctatgggagattaaaaccagagagaaagaagatttattatgaaccgcaatatgggaggaacaaagacaaccactgggatcagctggtgtcagtccctacttaggaaatactcagcaactgttagctgggaagaatgtattcggcaccttcccctgaggacctttctgaggagtaaaaagactactggcctctgtgccatggatgattcttttcccatcaccagaaatgatagcgtgcagtagagagcaaagatggctt

SEQ ID NO: 2 - protein, 822 aa, Homo sapiens
  ref <308>: DATABASE ACCESSION NUMBER: NCBI/NM_006180
  ref <309>: DATABASE ENTRY DATE: 2000-11-01
  ref <313>: RELEVANT RESIDUES: (1)..(822)
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 3 - DNA, 1870 bp, Homo sapiens
  ref <308>: DATABASE ACCESSION NUMBER: NCBI/S76474
  ref <309>: DATABASE ENTRY DATE: 1995-07-25
  ref <313>: RELEVANT RESIDUES: (1)..(1870)
ggaaggtttaaagaagaagccgcaaagcgcagggaaggcctcccggcacgggtgggggaaagcggccggtgcagcgcggggacaggcactcgggctggcactggctgctagggatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaacccaaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggttttgttttgtttcataagatcccactggatgggtagctgaaataaaggaaaagacagagaaaggggctgtggtgcttgttggttgatgctgccatgtaagctggactcctgggactgctgttggcttatcccgggaagtgctgcttatctggggttttctggtagatgtgggcggtgtttggaggctgtactatatgaagcctgcatatactgtgagctgtgattggggaacaccaatgcagaggtaactctcaggcagctaagcagcacctcaagaaaacatgttaaattaatgcttctcttcttacagtagttcaaatacaaaactgaaatgaaatcccattggattgtacttctct

SEQ ID NO: 4 - protein, 477 aa, Homo sapiens
  ref <308>: DATABASE ACCESSION NUMBER: NCBI/S76474
  ref <309>: DATABASE ENTRY DATE: 1995-07-25
  ref <313>: RELEVANT RESIDUES: (1)..(477)
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGFVLFHKIPLDG

SEQ ID NO: 5 - DNA, 8192 bp, Homo sapiens
  ref <308>: DATABASE ACCESSION NUMBER: NCBI/AF410900
  ref <309>: DATABASE ENTRY DATE: 2002-01-25
gggagcaggagcctcgctggctgcttcgctcgcgctctacgcgctcagtccccggcggtagcaggagcctggacccaggcgccggcggcgggcgtgaggcgccggagcccggcctcgaggtgcataccggacccccattcgcatctaacaaggaatctgcgccccagagagtcccggacgccgccggtcggtgcccggcgcgccgggccatgcagcgacggccgccgcggagctccgagcagcggtagcgcccccctgtaaagcggttcgctatgccgggaccactgtgaaccctgccgcctgccggaacactcttcgctccggaccagctcagcctctgataagctggactcggcacgcccgcaacaagcaccgaggagttaagagagccgcaagcgcagggaaggcctccccgcacgggtgggggaaagcggccggtgcagcgcggggacaggcactcgggctggcactggctgctagggatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatggcccagaggttcccccaagaccgcctgataataatttggtatttggaggctcctgtgtcactgcaggaactaaaggaggctaaatccatgcctgatggaggagaagagttctatggttatctgcaaattctggccagacaacatcttgacgtcactccttagcttccataacctagccaagcaagaagttgcctttccaagacaaagcagtgtgctctaatgactaacccctcaaagtactatgccactttaactatagacccatctcctcgatcaatcaggatggcaagatggagctgaggagctcagcaacatcaagtctggagttggtctttaactcaactagctcgtttagacgtgtctgaacaccacatcacctgacagcacggggtggtttcccagtaaaatttacaaactcagctcaagggcagctgtgttgctttcctttccttgactgctgagaaactttttgacagggaacaatggaaacacaccttctgagctgaaacaaacaaacagaaacaaaacatactaaccagcaaaatccccaaatcatcaatcttgggttctcttgaagggcaggagtgtgttttatcttctcccgtcggagcaaacactatagatgtcctccctaaaattctgtcttccctagagcagccttgtaaattagctagggtcctagggttgaggcctaaatcaacttaaaattgtctctaaatatgtacctggatgtgtttgtacttgcagagcatgccctcttcatgtgcctagggctagtaactccctgtggcagaggcatgtaaagtattctgactttttttttttcaacttaattccatttccaatgaaatggatttttaaaaattttctccagagtgtgccatacttctccagctattatagttaatgtgtgtgtatccttgtgtatatgtgtgtttgtgtgtgcatatgtgttttcctagtggttacatgcttactaggcaattatgtaaataagcacagattcataggccagctaggcctgaggaaagaagacattataaagggagggagtattttaacattagctaaagctatcacacaaggcacccattctgctcccctcaacagccacagcccacttcgtccttgtcttaccaataaggggaaaggctggaggtgatatttttcacagaaccgcagaggttttgaacatatttgcaacattactttgagtacacatgagcaaaaattctgaattacatccaggaccccagaagctcattagatcaaagagtgcggggcccctcagagttaccagagattatctgcagacttcagtgcaatcgaatgaccatggtccattttgatggtcagaggtaggactgaaaaacgggtagaaacaattgctttagcgcttccttctgtactttgcctattaatgttttgtctttcaaaaatatattttctcctaattgtttaattggccaaataatggctgctttgggagttgtttgtatgccttggaaggccatggcctgcactttaaaaataagctaagtccattctgcccagcacgagcattaggacagagaatgcacttattttaggatccttaaaaattgcttcttttatggcacactgggttgacgactcatctcgtgggagccttcatggcacattgctgctgttctgcaggtcccaatacaattccttccccctctcagtgccacggcccccccattgctagctacacaatttgatatcatattcccttttcaactccaaaggagatgataagaagctatcaaataatgctttaaaaaagcaacttgagtttcttaaaagaaaggaaatgaatacatgctgcataattacatttaaaatgtaagccatgttattataagccgcactgagatgaagatttgttagcaaaccagtttcaagcacactcacagtgaagtaaaatcatgtttttagcatctgaccattgggtaatattattctttgttatcaaaagagaaatatcacccaagtatagtatacttagacctcctagaggaaacactccagtcctaagcttggtgtctgaaaagaaaaacaaaaataaagattatggatttaggtcagggagacagagtgatattctgaagactgtgtttactccctcatcatcggccaaccaagatggagttctgcatcctgcacatatcagacatttcagtccaatttcaccaaagcatcagtgatgttctagaagcatcccagcagatggaggatcctaatgtatttgttctgggtatttcccaaggcccagcctgactggagtgtgtgtaccaacaggatgaatccaatcaagctacgcccccattttggtttcggattggccactcttgcatgtgctagtagattgtggaccaggaccagctgagcaaacacagttgcagagtagcctcctatgttgctaagaagctcctgctacccaggtgctttgaacaattgagtgctccctctggttaagtagagatggcaccaccggagtttttcttggatgtgaggctcaatcctttacggcagctattataacaaagtgaaggttttctccctgggaaatgcagcttttctctgtctttactaattctgccagcctgtgagagtaaccaccgtagctgggcttcttctcagattaattgtcatgccaggtctccttcctggggagctgtgatgctgctctgaggttgattgctgaggttgtagtgggtttttgtttgtttttgtttagtttttcttgattgttcttctttctcttgaatggcaagagaagaaacactttctctaacccacggccaggaaggaaatggggagagagctacttcttagttcaacctggttgccacataaaggaatctctctccttggactcagcccctaactggaagcaagagccactgccctctgagactgagagagcagcccgaggaggagatgaatccattctgccctttgtttgggtttgcttcctgtcagtgagagaatgctgaggcagttcctgttatgtgaaactttcatttttaaaaccaggacagtcctaaacagactggaatgagttggtcaatcccagttggtataggcccaatgatttttgctagtaagataggattgtcttcctcacccaaaatgccttcaagtgccctaaaatgggtattttaaaataagaataaataatgtagatttagtagaaaacctggaaaacataagaaacaaagatgaaacgaaaagtcccatgtaattccaccagttagagttaaccactgatatcgtttggatatatggctttctagtcttgtggatatccttttaatctcttgtaatataaagtctgaccatatgtgtccttgcatttgtttgtactggactctgttaatatttctatagtaatggctcactttggggagattgtgctgcacagtgtgtaggaagcacattgggtgtattattcccagttttgtattttgtatttccttggagatgtgcaggggttaagagcgggggtctggccatagctggccacgtcagactctcatatggtaagtatcacagagcacatgaggcctgtgttatgcgctggaaagactcaggaaatgagaggctctcttgttctgacaaggcaggctgagagctctcatttagggtcatcactccagataactccaaatgcagtttattgctcaactgaagcagatgatcactttttgcctccaagttcttcaccctagctagctcctttcaaagagccgagtatgctggatcttaaagggccaaactagttacatctcatacatttcctgatgtttagggatgccttcacttccatcaaggataccttggctgtgcaaggacctctgatagctggagtctccttttggtcactcccagctttgcttaaacttgatggagtttgctgtccagtgatccccggatctttcatcatgaaagccttccttcctctcctgatgtctcaggcctctagacctagactggggttctggcaaggaggcctctatcaatagtatgacatccaataatatgttagtgttgatattttgcacagtaatattaagtttaagagattataaaaatgagttcaaatgaataagttcctgtgatgtaagagattagatatgtgtgatttcagaaccaaagccaggggggaatcccagaaagaaaacaataatataatcctagtttctatatattatttttattcattactgtatatgggtagagatcaatattctttcttatgctgttactattaattaacacattttttaaccatgccattgaacttttgggtgcattaaagtggaacccaagctcctcattagataataatggcatttggactgagtgccatattcctaaatttccaataaagtggttgatatagagaggacaggataaagccctatagtgtgcagttatatcaaaacagctagtctccactttagggaatgcctttactagagattacatgaaatgtctgcttataaaataagcagagatggtaccactaagcagccacctgaattgttttcctacaggaatgattacttttcagatccatttatgttttcatgctcaatacttactccccttccctgcaacacccaaagagtttacttttgcaagtcatttggtcttcagtctactactgaggaatagagaggcactaactgctttacccaggatcagaactcatgttcttaccttctattaatagagtacttgagccagatggactaactggtctcacattttctctatcttggttttacttccataaacatcaatatctttacccacatgatttttccatcctcccatttttttccatatgtattagggttcaggaactatgatgctaatgatcacatttcttcctagttcctaatttcattagtgccatttcctgatatctacagaaacaattatcaatacatgtagctgcttgagccttatttagaaggctagcctttcttttccaagtgctgtcagaatgtatacatttagtctgtctttttcccttttaggagtctttgttctgggttgatggcaaaattcctctttttacatgtgagatttttgatttcactgaattctacctagatttttatggacattggattttaaagaggaaaacactcattttcttagtaagatattggtgatacatagctatgccattgatttccatactcctgagctttggggagggagacagtggccaagtagcaggcagaataagatcatcactcatgtcctgaatcaatcacactttccttctcggattgtgtatatgctctgccacttcctacatattacatcctgagtttttaagtaaagtggatcttagccagatttgagtctaatggctgattcatcggcatagttcttggcgttaacatctcagtgtcctctttagttctctttgaggattcatgtcattgagggcctttgtgcctccacttgtctcagtatgaggaagaactttggtgtgagggcggagctatgtgaagggttgctgggttgggggattagttcatatggtccccatgccatctatttacttttggagagaggggactttgagtgggtgggtatggatagatgttcctcaaggaaaccctgctggctaatgggcactacatctgtgtattactgtgattctctctgtaagctccccatgtggccaaggacccccctcctaccagggcacttcctgccacctcattgcactggtctcaaccattcagcctgctgctgctgcaccatgttgggctgcggtaggatagggaaggggttctgttgattgctaaatgttgcctaactttatttccctctcccacatttcatgcaagggagcggacctaacacatgacttgcattctcttcctatgttcagaaactccagggcttgcccacgtgtatgtatgagtgaccaatggagcttggaattctttatctatatgatctgtccgaaaatgagatcttttgtactggaatttgtgatgtagttgatcattcagagccaaacgcatataccaataaagacaagactgtcatataaaaaaaaaaaaaaaaa

SEQ ID NO: 6 - protein, 537 aa, Homo sapiens
  ref <308>: DATABASE ACCESSION NUMBER: NCBI/AF410900
  ref <309>: DATABASE ENTRY DATE: 2002-01-25
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTWPRGSPKTA

SEQ ID NO: 7 - DNA, 8240 bp, Homo sapiens
  ref <308>: DATABASE ACCESSION NUMBER: NCBI/AF410901
  ref <309>: DATABASE ENTRY DATE: 2002-01-25
gggagcaggagcctcgctggctgcttcgctcgcgctctacgcgctcagtccccggcggtagcaggagcctggacccaggcgccggcggcgggcgtgaggcgccggagcccggcctcgaggtgcataccggacccccattcgcatctaacaaggaatctgcgccccagagagtcccggacgccgccggtcggtgcccggcgcgccgggccatgcagcgacggccgccgcggagctccgagcagcggtagcgcccccctgtaaagcggttcgctatgccgggaccactgtgaaccctgccgcctgccggaacactcttcgctccggaccagctcagcctctgataagctggactcggcacgcccgcaacaagcaccgaggagttaagagagccgcaagcgcagggaaggcctccccgcacgggtgggggaaagcggccggtgcagcgcggggacaggcactcgggctggcactggctgctagggatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaagatttctcatggtttggatttgggaaagtaaaatcaagacaaggtgttggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatggcccagaggttcccccaagaccgcctgataataatttggtatttggaggctcctgtgtcactgcaggaactaaaggaggctaaatccatgcctgatggaggagaagagttctatggttatctgcaaattctggccagacaacatcttgacgtcactccttagcttccataacctagccaagcaagaagttgcctttccaagacaaagcagtgtgctctaatgactaacccctcaaagtactatgccactttaactatagacccatctcctcgatcaatcaggatggcaagatggagctgaggagctcagcaacatcaagtctggagttggtctttaactcaactagctcgtttagacgtgtctgaacaccacatcacctgacagcacggggtggtttcccagtaaaatttacaaactcagctcaagggcagctgtgttgctttcctttccttgactgctgagaaactttttgacagggaacaatggaaacacaccttctgagctgaaacaaacaaacagaaacaaaacatactaaccagcaaaatccccaaatcatcaatcttgggttctcttgaagggcaggagtgtgttttatcttctcccgtcggagcaaacactatagatgtcctccctaaaattctgtcttccctagagcagccttgtaaattagctagggtcctagggttgaggcctaaatcaacttaaaattgtctctaaatatgtacctggatgtgtttgtacttgcagagcatgccctcttcatgtgcctagggctagtaactccctgtggcagaggcatgtaaagtattctgactttttttttttcaacttaattccatttccaatgaaatggatttttaaaaattttctccagagtgtgccatacttctccagctattatagttaatgtgtgtgtatccttgtgtatatgtgtgtttgtgtgtgcatatgtgttttcctagtggttacatgcttactaggcaattatgtaaataagcacagattcataggccagctaggcctgaggaaagaagacattataaagggagggagtattttaacattagctaaagctatcacacaaggcacccattctgctcccctcaacagccacagcccacttcgtccttgtcttaccaataaggggaaaggctggaggtgatatttttcacagaaccgcagaggttttgaacatatttgcaacattactttgagtacacatgagcaaaaattctgaattacatccaggaccccagaagctcattagatcaaagagtgcggggcccctcagagttaccagagattatctgcagacttcagtgcaatcgaatgaccatggtccattttgatggtcagaggtaggactgaaaaacgggtagaaacaattgctttagcgcttccttctgtactttgcctattaatgttttgtctttcaaaaatatattttctcctaattgtttaattggccaaataatggctgctttgggagttgtttgtatgccttggaaggccatggcctgcactttaaaaataagctaagtccattctgcccagcacgagcattaggacagagaatgcacttattttaggatccttaaaaattgcttcttttatggcacactgggttgacgactcatctcgtgggagccttcatggcacattgctgctgttctgcaggtcccaatacaattccttccccctctcagtgccacggcccccccattgctagctacacaatttgatatcatattcccttttcaactccaaaggagatgataagaagctatcaaataatgctttaaaaaagcaacttgagtttcttaaaagaaaggaaatgaatacatgctgcataattacatttaaaatgtaagccatgttattataagccgcactgagatgaagatttgttagcaaaccagtttcaagcacactcacagtgaagtaaaatcatgtttttagcatctgaccattgggtaatattattctttgttatcaaaagagaaatatcacccaagtatagtatacttagacctcctagaggaaacactccagtcctaagcttggtgtctgaaaagaaaaacaaaaataaagattatggatttaggtcagggagacagagtgatattctgaagactgtgtttactccctcatcatcggccaaccaagatggagttctgcatcctgcacatatcagacatttcagtccaatttcaccaaagcatcagtgatgttctagaagcatcccagcagatggaggatcctaatgtatttgttctgggtatttcccaaggcccagcctgactggagtgtgtgtaccaacaggatgaatccaatcaagctacgcccccattttggtttcggattggccactcttgcatgtgctagtagattgtggaccaggaccagctgagcaaacacagttgcagagtagcctcctatgttgctaagaagctcctgctacccaggtgctttgaacaattgagtgctccctctggttaagtagagatggcaccaccggagtttttcttggatgtgaggctcaatcctttacggcagctattataacaaagtgaaggttttctccctgggaaatgcagcttttctctgtctttactaattctgccagcctgtgagagtaaccaccgtagctgggcttcttctcagattaattgtcatgccaggtctccttcctggggagctgtgatgctgctctgaggttgattgctgaggttgtagtgggtttttgtttgtttttgtttagtttttcttgattgttcttctttctcttgaatggcaagagaagaaacactttctctaacccacggccaggaaggaaatggggagagagctacttcttagttcaacctggttgccacataaaggaatctctctccttggactcagcccctaactggaagcaagagccactgccctctgagactgagagagcagcccgaggaggagatgaatccattctgccctttgtttgggtttgcttcctgtcagtgagagaatgctgaggcagttcctgttatgtgaaactttcatttttaaaaccaggacagtcctaaacagactggaatgagttggtcaatcccagttggtataggcccaatgatttttgctagtaagataggattgtcttcctcacccaaaatgccttcaagtgccctaaaatgggtattttaaaataagaataaataatgtagatttagtagaaaacctggaaaacataagaaacaaagatgaaacgaaaagtcccatgtaattccaccagttagagttaaccactgatatcgtttggatatatggctttctagtcttgtggatatccttttaatctcttgtaatataaagtctgaccatatgtgtccttgcatttgtttgtactggactctgttaatatttctatagtaatggctcactttggggagattgtgctgcacagtgtgtaggaagcacattgggtgtattattcccagttttgtattttgtatttccttggagatgtgcaggggttaagagcgggggtctggccatagctggccacgtcagactctcatatggtaagtatcacagagcacatgaggcctgtgttatgcgctggaaagactcaggaaatgagaggctctcttgttctgacaaggcaggctgagagctctcatttagggtcatcactccagataactccaaatgcagtttattgctcaactgaagcagatgatcactttttgcctccaagttcttcaccctagctagctcctttcaaagagccgagtatgctggatcttaaagggccaaactagttacatctcatacatttcctgatgtttagggatgccttcacttccatcaaggataccttggctgtgcaaggacctctgatagctggagtctccttttggtcactcccagctttgcttaaacttgatggagtttgctgtccagtgatccccggatctttcatcatgaaagccttccttcctctcctgatgtctcaggcctctagacctagactggggttctggcaaggaggcctctatcaatagtatgacatccaataatatgttagtgttgatattttgcacagtaatattaagtttaagagattataaaaatgagttcaaatgaataagttcctgtgatgtaagagattagatatgtgtgatttcagaaccaaagccaggggggaatcccagaaagaaaacaataatataatcctagtttctatatattatttttattcattactgtatatgggtagagatcaatattctttcttatgctgttactattaattaacacattttttaaccatgccattgaacttttgggtgcattaaagtggaacccaagctcctcattagataataatggcatttggactgagtgccatattcctaaatttccaataaagtggttgatatagagaggacaggataaagccctatagtgtgcagttatatcaaaacagctagtctccactttagggaatgcctttactagagattacatgaaatgtctgcttataaaataagcagagatggtaccactaagcagccacctgaattgttttcctacaggaatgattacttttcagatccatttatgttttcatgctcaatacttactccccttccctgcaacacccaaagagtttacttttgcaagtcatttggtcttcagtctactactgaggaatagagaggcactaactgctttacccaggatcagaactcatgttcttaccttctattaatagagtacttgagccagatggactaactggtctcacattttctctatcttggttttacttccataaacatcaatatctttacccacatgatttttccatcctcccatttttttccatatgtattagggttcaggaactatgatgctaatgatcacatttcttcctagttcctaatttcattagtgccatttcctgatatctacagaaacaattatcaatacatgtagctgcttgagccttatttagaaggctagcctttcttttccaagtgctgtcagaatgtatacatttagtctgtctttttcccttttaggagtctttgttctgggttgatggcaaaattcctctttttacatgtgagatttttgatttcactgaattctacctagatttttatggacattggattttaaagaggaaaacactcattttcttagtaagatattggtgatacatagctatgccattgatttccatactcctgagctttggggagggagacagtggccaagtagcaggcagaataagatcatcactcatgtcctgaatcaatcacactttccttctcggattgtgtatatgctctgccacttcctacatattacatcctgagtttttaagtaaagtggatcttagccagatttgagtctaatggctgattcatcggcatagttcttggcgttaacatctcagtgtcctctttagttctctttgaggattcatgtcattgagggcctttgtgcctccacttgtctcagtatgaggaagaactttggtgtgagggcggagctatgtgaagggttgctgggttgggggattagttcatatggtccccatgccatctatttacttttggagagaggggactttgagtgggtgggtatggatagatgttcctcaaggaaaccctgctggctaatgggcactacatctgtgtattactgtgattctctctgtaagctccccatgtggccaaggacccccctcctaccagggcacttcctgccacctcattgcactggtctcaaccattcagcctgctgctgctgcaccatgttgggctgcggtaggatagggaaggggttctgttgattgctaaatgttgcctaactttatttccctctcccacatttcatgcaagggagcggacctaacacatgacttgcattctcttcctatgttcagaaactccagggcttgcccacgtgtatgtatgagtgaccaatggagcttggaattctttatctatatgatctgtccgaaaatgagatcttttgtactggaatttgtgatgtagttgatcattcagagccaaacgcatataccaataaagacaagactgtcatataaaaaaaaaaaaaaaaa

SEQ ID NO: 8 - protein, 553 aa, Homo sapiens
  ref <308>: DATABASE ACCESSION NUMBER: NCBI/AF410901
  ref <309>: DATABASE ENTRY DATE: 2002-01-25
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKDFSWFGFGKVKSRQGVGPASVISNDDDSASPLHHISNGSNTPSSSEGGPDAVIIGMTKIPVIENPQYFGITNSQLKPDTWPRGSPKTA

SEQ ID NO: 9 - DNA, 4351 bp, Mus musculus
  ref <308>: DATABASE ACCESSION NUMBER: NCBI/X17647
  ref <309>: DATABASE ENTRY DATE: 1995-03-22
  ref <313>: RELEVANT RESIDUES: (1)..(4351)
aggctgcggcggcggcgcgccgttagagcccagtcgctgcttcagctgctgttgctgcttctgccaggctctgctccctgcgcttgctacgggaggccggggaagccgcgcggacagtcctcggtggcctgggccggcactgtcctgctaccgcagttgctccccagccctgaggtgcgcaccgatatcgatattcgtgccggtttagcggttctgcgacccaaagagtccagggagatccaccgagtggtgcctggtgtataggactatgcagccgccttgtggctcggagcagcggcccgcgatgtcccagccactgtgaaccatttggtcagcgccaacctgctcagccccagcaccgacaggctcagcctctggtacgctccactccgcgggaggccaccagcaccaagcagcaagagggcgcagggaaggcctcccccctccggcgggggacgcctggctcagcgtagggacacgcactccgactgactggcactggcagctcgggatgtcgccctggctgaagtggcatggacccgccatggcgcggctctggggcttatgcctgctggtcttgggcttctggagggcctctctcgcctgcccgacgtcctgcaaatgcagttccgctaggatttggtgtactgagccttctccaggcatcgtggcattcccgaggttggaacctaacagcgttgacccggagaacatcacggaaattctcattgcaaaccagaaaaggctagaaatcatcaatgaagatgacgttgaagcttacgtggggctgagaaaccttacaattgtggattccggcttaaagtttgtggcttacaaagcgtttctgaaaaacagcaacctgcggcacataaatttcacacgaaacaagctgacgagtttgtccaggagacatttccgccaccttgacttgtctgacctgatcctgacgggtaatccgttcacgtgctcctgcgacatcatgtggctcaagactctccaggagactaaatccagccccgacactcaggatttgtactgcctcaatgagagcagcaagaacatgcccctggcgaacctgcagatacccaattgtggtctgccatctgcacgtctggctgctcctaacctcaccgtggaggaaggaaagtctgtgaccctttcctgcagtgtggggggtgacccactccccaccttgtactgggacgttgggaatttggtttccaagcacatgaatgaaacaagccacacacagggctccttaaggataacgaacatttcatctgatgacagtggaaagcaaatctcttgtgtggcagaaaaccttgtaggagaagatcaagattctgtgaacctcactgtgcattttgcgccaactatcacgtttctcgagtctccaacctcagatcaccactggtgcattccattcactgtgagaggcaaccccaagcctgcgcttcagtggttctacaatggggccatactgaatgagtccaagtacatctgtactaagatccacgtcaccaatcacacggagtaccatggctgcctccagctggataaccccactcatatgaataacggagactacaccctgatggccaagaacgagtatgggaaggatgagagacagatctccgctcacttcatgggccggcctggagtcgactacgagacaaacccaaattaccctgaagtcctctatgaagactggaccacgccaactgacattggggatactacgaacaaaagtaatgaaatcccctccacggatgttgctgaccaaagcaatcgggagcatctctcggtctatgccgtggtggtgattgcatctgtggtgggattctgcctgctggtgatgttgctcctgctcaagttggcgagacattccaagtttggcatgaaaggcccagcttcggtcatcagcaacgacgatgactctgccagccccctccaccacatctccaatgggagtaacactccatcttcttcggagggcggtcccgacgctgtcattattggaatgaccaagattcctgttattgaaaacccccagtactttggcatcaccaacagtcagctcaagccagacacatttgttcagcacatcaagagacacaacatcgttctgaagagggaacttggggaaggagccttcgggaaagttttccttgccgagtgctacaacctctgcccagagcaggataagatcctggtggctgtgaagacgctgaaggacgccagcgacaatgcacgcaaggactttcatcgggaagctgagctgctgaccaacctccagcacgagcacattgtcaagttctacggtgtctgtgtggagggcgacccactcatcatggtctttgagtacatgaagcacggggacctcaacaagttccttagggcacacgggcccgacgcagtgctgatggcagagggtaacccgcccacagagctgacgcagtcgcagatgctgcacatcgctcagcaaatcgcagcaggtatggtctacctggcgtcccaacactttgtgcaccgtgacctggccacccggaactgcctggtgggagagaacctgctggtgaaaattggggactttgggatgtcccgagatgtgtacagcaccgactactatcgggtcggtggccacacaatgttgcccatccgatggatgcctccagagagcatcatgtataggaaattcaccaccgagagcgacgtctggagcctgggcgttgtgttgtgggagatcttcacctacggcaagcagccctggtatcagctatcgaacaatgaggtgatagagtgcatcacccagggaagagtccttcagcggcctcgaacctgtccccaggaggtgtatgagctcatgctcggatgctggcagcgggaaccacacacccggaagaacatcaagagcatccacaccctccttcagaacttggccaaggcatctcccgtctacctggatatcctaggctagggtcctccttctgcccagaccgtccttcccaaggccctcctcagactggtcctcagactggcctacacgacgaacctcttgactgccgctgacgtcatgaccttgctgtccttcgctctgacagagttgacaggaccaggagcggctctctgggggaggcagtgtgtgcttctccatccacagacagtattaactcgcttctggcatcgtctctttctctcccttgggtttgtttctttcttttgccccttccccttttatcattatttattcatttatttattttctggtcttcacgcttcacggccctcagtctctccttgaccaatctggcttctgcattcctattaactgtacatagacaaaggccttaacaaacctaatttgttatatcagcagacactccagtttgcccaccacaactaacaatgccttgttgtattcctgcctttgacgtggatgaaaaaaagagaaaaaaggccgagactctcctgcaggaatcggatgaggcctctgagctcaagcccgtggaactggacacttttgaaggaaacatcacaaagcaactggtgaagaggctcacctcggctgaggggcccgtcactactgacaagcttttctttgaaggctctgttggtagcgagtctgaggctggccggtcctttctggatggcagcctggaagatgccttcaatggactcttccttgcattagacccacacaagaagcagtacaaagagttccaggatctgaaccaagaagtcactcacttggatgatgttctcaaagatgctaaacatcttgaggatcagagactcaatgatgctgcttcccggatggagatcacagagggtgaatgagacaaccgagatttaaaagactgaaggacattttcccatgtgcttctgtgtcatcccaagtgtctgggacagatccccgcaaggcccttcctaccttgtgctaagagtctgcaaggggatcctcctagccagacagaggacacgcaggtgtctcctttgcaagatttgtcctgttcaacctacctcacgtcctcttgaatatgtggatatgcttttctttctccaggctaaagcactggcatagcagccacatagcaggcttctgtgttggctcatgtcctgcaaacctgctgtagaaggaacttgtccccataattccagggcttgcccgaggggtgatgggacttgtgcctttcaccttcaggggagtcgggatcattgtcccatcatgcccaagtcacccatttgcctctccgtgctcagaaaaaaaagcatccttgaatggaacatggtgatgcagggctccgtgccaaagcagcctagggcaggtgtatttgagcagtttccttttctg

SEQ ID NO: 10 - protein, 821 aa, Mus musculus
  ref <308>: DATABASE ACCESSION NUMBER: NCBI/X17647
  ref <309>: DATABASE ENTRY DATE: 1995-03-22
  ref <313>: RELEVANT RESIDUES: (1)..(821)
MSPWLKWHGPAMARLWGLCLLVLGFWRASLACPTSCKCSSARIWCTEPSPGIVAFPRLEPNSVDPENITEILIANQKRLEIINEDDVEAYVGLRNLTIVDSGLKFVAYKAFLKNSNLRHINFTRNKLTSLSRRHFRHLDLSDLILTGNPFTCSCDIMWLKTLQETKSSPDTQDLYCLNESSKNMPLANLQIPNCGLPSARLAAPNLTVEEGKSVTLSCSVGGDPLPTLYWDVGNLVSKHMNETSHTQGSLRITNISSDDSGKQISCVAENLVGEDQDSVNLTVHFAPTITFLESPTSDHHWCIPFTVRGNPKPALQWFYNGAILNESKYICTKIHVTNHTEYHGCLQLDNPTHMNNGDYTLMAKNEYGKDERQISAHFMGRPGVDYETNPNYPEVLYEDWTTPTDIGDTTNKSNEIPSTDVADQSNREHLSVYAVVVIASVVGFCLLVMLLLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHTRKNIKSIHTLLQNLAKASPVYLDILG

SEQ ID NO: 11 - DNA, 2484 bp, Mus musculus
  ref <308>: DATABASE ACCESSION NUMBER: NCBI/M33385
  ref <309>: DATABASE ENTRY DATE: 1993-04-23
  ref <313>: RELEVANT RESIDUES: (1)..(2484)
atgtcgccctggctgaagtggcatggacccgccatggcgcggctctggggcttatgcctgctggtcttgggcttctggagggcctctctcgcctgcccgacgtcctgcaaatgcagttccgctaggatttggtgtactgagccttctccaggcatcgtggcattcccgaggttggaacctaacagcgttgacccggagaacatcacggaaattctcattgcaaaccagaaaaggctagaaatcatcaatgaagatgacgttgaagcttacgtggggctgagaaaccttacaattgtggattccggcttaaagtttgtggcttacaaagcgtttctgaaaaacagcaacctgcggcacataaatttcacacgaaacaagctgacgagtttgtccaggagacatttccgccaccttgacttgtctgacctgatcctgacgggtaatccgttcacgtgctcctgcgacatcatgtggctcaagactctccaggagactaaatccagccccgacactcaggatttgtactgcctcaatgagagcagcaagaacatgcccctggcgaacctgcagatacccaattgtggtctgccatctgcacgtctggctgctcctaacctcaccgtggaggaaggaaagtctgtgaccctttcctgcagtgtggggggtgacccactccccaccttgtactgggacgttgggaatttggtttccaagcacatgaatgaaacaagccacacacagggctccttaaggataacgaacatttcatctgatgacagtggaaagcaaatctcttgtgtggcagaaaaccttgtaggagaagatcaagattctgtgaacctcactgtgcattttgcgccaactatcacgtttctcgagtctccaacctcagatcaccactggtgcattccattcactgtgagaggcaaccccaagcctgcgcttcagtggttctacaatggggccatactgaatgagtccaagtacatctgtactaagatccacgtcaccaatcacacggagtaccatggctgcctccagctggataaccccactcatatgaataacggagactacaccctgatggccaagaacgagtatgggaaggatgagagacagatctccgctcacttcatgggccggcctggagtcgactacgagacaaacccaaattaccctgaagtcctctatgaagactggaccacgccaactgacattggggatactacgaacaaaagtaatgaaatcccctccacggatgttgctgaccaaagcaatcgggagcatctctcggtctatgccgtggtggtgattgcatctgtggtgggattctgcctgctggtgatgttgctcctgctcaagttggcgagacattccaagtttggcatgaaaggttttgttttgtttcataagatcccactggatgggtagctgagataaaggaaagacaaaggctggggctgtggtgcttgttgcctgacgccctgtgagctgaactctgggactgctgttgcctatcccaggaagtgctgcttatttgagggtgtctggtggaaatgggtaatctccgaggatgtctgcagcctgcttgttgtgagctgtgactggggaaccccaaggcagaggcaggggtcaggcagctgagaagcagcagaagaacacacttagattcaccttctgttcttacaatagttcaaatatagaatcgaagtgaaatctcattggattatgcctctctaatgaaaagcgagctgtttgactatacggaaaatgtgctgacattaattgcttctgtttattaaaggtgatttgcaaattaaaaactctgcatctatcatctatccatctatctgtttgtctatcatatctatctgtctgtctatctgtctatcatctatctacctacctctctatcatatctatctgtctgtctatctatctatctatctatctatctatctatctatctatctatctatctatctatcatctatctacctatcatcgatctacttatctatcatctatctatctacctatcatcgatttacttatctatcatctatctatctatctatctatctatctatctatctatctatctgtcatctatctaaagtcatagctaggtctaagtgcacactaaaagtctaatccacacataacacctatttcagcaacatcttctgttctctaacctttgctaacttctgtgatttccacctacaaccctgcgactgatagacttaaaggcacattggtggtgtcattagtaggttctttgttttgctggcagcaaagacccaaactcttcgctaacgattgctttcaaagtccacccggcaggtagaacggagcagcaccagggactgtgtggccaggagtatggacctgaattaaccacagcctgagaataaataatggtagggtatatgcatatagggaattaaaatcttgtccctttccattgccctctgctaaccg

SEQ ID NO: 12 - protein, 476 aa, Mus musculus
  ref <308>: DATABASE ACCESSION NUMBER: NCBI/M33385
  ref <309>: DATABASE ENTRY DATE: 1993-04-27
  ref <313>: RELEVANT RESIDUES: (1)..(476)
MSPWLKWHGPAMARLWGLCLLVLGFWRASLACPTSCKCSSARIWCTEPSPGIVAFPRLEPNSVDPENITEILIANQKRLEIINEDDVEAYVGLRNLTIVDSGLKFVAYKAFLKNSNLRHINFTRNKLTSLSRRHFRHLDLSDLILTGNPFTCSCDIMWLKTLQETKSSPDTQDLYCLNESSKNMPLANLQIPNCGLPSARLAAPNLTVEEGKSVTLSCSVGGDPLPTLYWDVGNLVSKHMNETSHTQGSLRITNISSDDSGKQISCVAENLVGEDQDSVNLTVHFAPTITFLESPTSDHHWCIPFTVRGNPKPALQWFYNGAILNESKYICTKIHVTNHTEYHGCLQLDNPTHMNNGDYTLMAKNEYGKDERQISAHFMGRPGVDYETNPNYPEVLYEDWTTPTDIGDTTNKSNEIPSTDVADQSNREHLSVYAVVVIASVVGFCLLVMLLLLKLARHSKFGMKGFVLFHKIPLDG

SEQ ID NO: 13 - DNA, 2838 bp, Homo sapiens
  ref <308>: DATABASE ACCESSION NUMBER: NCBI/XM_038336
  ref <309>: DATABASE ENTRY DATE: 2002-02-07
cgagccggccaccatgcccggcagaccgcgccactaggcgctcctcgcggctcccacccggcggcggcggcggcggcggcggcgtccgcgatggtttcagacgctgaaggattttgcatctgatcgctcggcgtttcaaagaagcagcgatcggagatggatgtctctctttgcccagccaagtgtagtttctggcggattttcttgctgggaagcgtctggctggactatgtgggctccgtgctggcttgccctgcaaattgtgtctgcagcaagactgagatcaattgccggcggccggacgatgggaacctcttccccctcctggaagggcaggattcagggaacagcaatgggaacgccagtatcaacatcacggacatctcaaggaatatcacttccatacacatagagaactggcgcagtcttcacacgctcaacgccgtggacatggagctctacaccggacttcaaaagctgaccatcaagaactcaggacttcggagcattcagcccagagcctttgccaagaacccccatttgcgttatataaacctgtcaagtaaccggctcaccacactctcgtggcagctcttccagacgctgagtcttcgggaattgcagttggagcagaactttttcaactgcagctgtgacatccgctggatgcagctctggcaggagcagggggaggccaagctcaacagccagaacctctactgcatcaacgctgatggctcccagcttcctctcttccgcatgaacatcagtcagtgtgaccttcctgagatcagcgtgagccacgtcaacctgaccgtacgagagggtgacaacgctgttatcacttgcaatggctctggatcaccccttcctgatgtggactggatagtcactgggctgcagtccatcaacactcaccagaccaatctgaactggaccaatgttcatgccatcaacttgacgctggtgaatgtgacgagtgaggacaatggcttcaccctgacgtgcattgcagagaacgtggtgggcatgagcaatgccagtgttgccctcactgtctactatcccccacgtgtggtgagcctggaggagcctgagctgcgcctggagcactgcatcgagtttgtggtgcgtggcaaccccccaccaacgctgcactggctgcacaatgggcagcctctgcgggagtccaagatcatccatgtggaatactaccaagagggagagatttccgagggctgcctgctcttcaacaagcccacccactacaacaatggcaactataccctcattgccaaaaacccactgggcacagccaaccagaccatcaatggccacttcctcaaggagccctttccagagagcacggataactttatcttgtttgacgaagtgagtcccacacctcctatcactgtgacccacaaaccagaagaagacacttttggggtatccatagcagttggacttgctgcttttgcctgtgtcctgttggtggttctcttcgtcatgatcaacaaatatggtcgacggtccaaatttggaatgaagggtcccgtggctgtcatcagtggtgaggaggactcagccagcccactgcaccacatcaaccacggcatcaccacgccctcgtcactggatgccgggcccgacactgtggtcattggcatgactcgcatccctgtcattgagaacccccagtacttccgtcagggacacaactgccacaagccggacacgtatgtgcagcacattaagaggagagacatcgtgctgaagcgagaactgggtgagggagcctttggaaaggtcttcctggccgagtgctacaacctcagcccgaccaaggacaagatgcttgtggctgtgaaggccctgaaggatcccaccctggctgcccggaaggatttccagagggaggccgagctgctcaccaacctgcagcatgagcacattgtcaagttctatggagtgtgcggcgatggggaccccctcatcatggtctttgaatacatgaagcatggagacctgaataagttcctcagggcccatgggccagatgcaatgatccttgtggatggacagccacgccaggccaagggtgagctggggctctcccaaatgctccacattgccagtcagatcgcctcgggtatggtgtacctggcctcccagcactttgtgcaccgagacctggccaccaggaactgcctggttggagcgaatctgctagtgaagattggggacttcggcatgtccagagatgtctacagcacggattattacaggctctttaatccatctggaaatgatttttgtatatggtgtgaggtgggaggacacaccatgctccccattcgctggatgcctcctgaaagcatcatgtaccggaagttcactacagagagtgatgtatggagcttcggggtgatcctctgggagatcttcacctatggaaagcagccatggttccaactctcaaacacggaggtcattgagtgcattacccaaggtcgtgttttggagcggccccgagtctgccccaaagaggtgtacgatgtcatgctggggtgctggcagagggaaccacagcagcggttgaacatcaaggagatctacaaaatcctccatgctttggggaaggccaccccaatctacctggacattcttggctagtggtggctggtggtcatgaattcatactctgttgcctcctctctccctgcctcacatctcccttccacctcacaactccttccatccttgactgaagcgaacatcttcatataaactcaagtgcctgctacacatacaacactgaaaaaaggaaaaaaaaag

SEQ ID NO: 14 - protein, 839 aa, Homo sapiens
  ref <308>: DATABASE ACCESSION NUMBER: NCBI/XM_038336
  ref <309>: DATABASE ENTRY DATE: 2002-02-07
MDVSLCPAKCSFWRIFLLGSVWLDYVGSVLACPANCVCSKTEINCRRPDDGNLFPLLEGQDSGNSNGNASINITDISRNITSIHIENWRSLHTLNAVDMELYTGLQKLTIKNSGLRSIQPRAFAKNPHLRYINLSSNRLTTLSWQLFQTLSLRELQLEQNFFNCSCDIRWMQLWQEQGEAKLNSQNLYCINADGSQLPLFRMNISQCDLPEISVSHVNLTVREGDNAVITCNGSGSPLPDVDWIVTGLQSINTHQTNLNWTNVHAINLTLVNVTSEDNGFTLTCIAENVVGMSNASVALTVYYPPRVVSLEEPELRLEHCIEFVVRGNPPPTLHWLHNGQPLRESKIIHVEYYQEGEISEGCLLFNKPTHYNNGNYTLIAKNPLGTANQTINGHFLKEPFPESTDNFILFDEVSPTPPITVTHKPEEDTFGVSIAVGLAAFACVLLVVLFVMINKYGRRSKFGMKGPVAVISGEEDSASPLHHINHGITTPSSLDAGPDTVVIGMTRIPVIENPQYFRQGHNCHKPDTYVQHIKRRDIVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRLFNPSGNDFCIWCEVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKILHALGKATPIYLDILG

SEQ ID NO: 15 - DNA, 1030 bp, Homo sapiens
  ref <308>: DATABASE ACCESSION NUMBER: NCBI/AJ224536
  ref <309>: DATABASE ENTRY DATE: 2000-11-29
ccgacacggatctttccagggcccacaaatgctgcatggtctccaaaggagatttcatcctcagaagctacaatgatatctctttatagaagttgtagtcttcaggtcttcagtgagctaacagcttttgtttttccaatggtttatgccctaacaatggcaaggaagattttaaggaaccaaacaccaccacctcctctcatctcctcatcatccccgccttgtcacattgctttcctcttgaaaattagctgaatttttttgatgggatattagaagccagaaagagggtcttgggtccaggattatctcccaagtcagaagaaacatccatccaggcccaggaatgacactctgaatggcaatgatgggcaccattttgagacattctggtccaagaaggaaaatgggggcaaatatgttaggaaaagtgcaggacagagttcatggtgatggtgaatctttcttctctgactctaacttgtgccatttctataatgccagggtgagattcttaggatctagattttatgcgtaaaataaaccagctgccactacaggcacagcagagtgggtacaggagctgagaaacctggatttctgtttctggcattgtgcacttaagaaaaatactttcccatgttttttgcacttggggtttaatactgaccattaattcccccatgtctgcctcttctgccaggggtcttttcaaacatagacaatcatgggatattaaacttgaaggacaatagagatcatctagtcccatcaactcactatatatatgaggaacctgaggtccagagtggggaagtgtcttacccaaggtcacatggtgagttacctcctttgacgtctttgtatgcagtaaagatccctcccctaaccaattttggttcttaagaccttaagactcatcaagcctccatatatttcgtggactgaggtacgactaggtgcccagcacgggatttggtactaaaaaaatcccttaaattaaaggagtgtcttccaggggaggaagctt

SEQ ID NO: 16 - DNA, 1113 bp, Homo sapiens
  ref <308>: DATABASE ACCESSION NUMBER: NCBI/AJ224537
  ref <309>: DATABASE ENTRY DATE: 2000-11-29
cagtggaggctgcagcaaaatggagtgtttccagtttctctgaccatgcagggttttaagttaatcccctcctcctatccttcccttttgctgacagttcttcccctttcaagctccctctctatttcccctcctagttttgatcttctttgggggttttggtttttactttattttgcttttttctgtttttttttctttttgttttttataggtttcagagaaattatgttgaatccaataagccttcccggacattccaagcctcttaaccatggcatctatgttgaggatgtcaatgtttatttcagcaaaggacgtcatggcttttaaaaactccttttaagcctccttgttttgatgtcaccttggtaggctgggccctctgagaggttggaagctctaggcattgttctctttggatccagggatgctaagtagaaactgcatgagccaccagtgccccggcaccctttaacaccaccagatgggtgttttcccccatccaccactggcagggttgccccttccctccaatcatcactgtgctccttttttcccggcctacgaggcagctcctgccactatctttagagccaataaagagaattaaaaacctgtgcaccaggagcatcttttaaatacactagccattctcttgctttacaaaaacaacctaaccatcacaagaaagcctgatgaagtccagccgtgctccagcctcactttccctgcttggaagcgtggggtctccctggctctcccaggataccatgctgtcctcttagtgacctcgtcgccctgcaacctccagtggggaagagtcacagagagcacctaagcagaggtggagacggcgcggtaagaggagggggagccaggctcaagtattggcaccaagttaggtctcagaggaaagaatggaaaccaatcactttacatttttatttttattttcggtggaaaaatcatccttttttgggacatacttgccccctacttcctcttctctctggaacggctcacaatgagtgtgacattagaaaactccttgcagaggagagtttctccaggctcttcctggggccttagatctgcagttccgacaagctt

SEQ ID NO: 17 - DNA, 1089 bp, Mus musculus
  misc_feature: Anti-sense RNA complementary to sequences specific for mouse TrkB.T1
cggttagcagagggcaatggaaagggacaagattttaattccctatatgcatataccctaccattatttattctcaggctgtggttaattcaggtccatactcctggccacacagtccctggtgctgctccgttctacctgccgggtggactttgaaagcaatcgttagcgaagagtttgggtctttgctgccagcaaaacaaagaacctactaatgacaccaccaatgtgcctttaagtctatcagtcgcagggttgtaggtggaaatcacagaagttagcaaaggttagagaacagaagatgttgctgaaataggtgttatgtgtggattagacttttagtgtgcacttagacctagctatgactttagatagatgacagatagatagatagatagatagatagatagatagatagatagatgatagataagtaaatcgatgataggtagatagatagatgatagataagtagatcgatgataggtagatagatgatagatagatagatagatagatagatagatagatagatagatagatagatagatagacagacagatagatatgatagagaggtaggtagatagatgatagacagatagacagacagatagatatgatagacaaacagatagatggatagatgatagatgcagagtttttaatttgcaaatcacctttaataaacagaagcaattaatgtcagcacattttccgtatagtcaaacagctcgcttttcattagagaggcataatccaatgagatttcacttcgattctatatttgaactattgtaagaacagaaggtgaatctaagtgtgttcttctgctgcttctcagctgcctgacccctgcctctgccttggggttccccagtcacagctcacaacaagcaggctgcagacatcctcggagattacccatttccaccagacaccctcaaataagcagcacttcctgggataggcaacagcagtcccagagttcagctcacagggcgtcaggcaacaagcaccacagccccagcctttgtctttcctttatctcagctacccatccagtgggatcttatgaaacaaaacaaa

SEQ ID NO: 18 - RNA, 20 nt, Mus musculus
  misc_feature: Anti-sense RNA for mouse TrkB.T1
aagcaggcugcagacauccu

SEQ ID NO: 19 - DNA, 359 bp, Homo sapiens
  misc_feature: Anti-sense RNA specific for human TrkB.T1
agagaagtacaatccaatgggatttcatttcagttttgtatttgaactactgtaagaagagaagcattaatttaacatgttttcttgaggtgctgcttagctgcctgagagttacctctgcattggtgttccccaatcacagctcacagtatatgcaggcttcatatagtacagcctccaaacaccgcccacatctaccagaaaaccccagataagcagcacttcccgggataagccaacagcagtcccaggagtccagcttacatggcagcatcaaccaacaagcaccacagcccctttctctgtcttttcctttatttcagctacccatccagtgggatcttatgaaacaaaacaaa

SEQ ID NO: 20 - DNA, 296 bp, Homo sapiens
  misc_feature: Anti-sense RNA complementary to Exon 19 of Human TrkB.Shc
ctccatcttgccatcctgattgatcgaggagatgggtctatagttaaagtggcatagtactttgaggggttagtcattagagcacactgctttgtcttggaaaggcaacttcttgcttggctaggttatggaagctaaggagtgacgtcaagatgttgtctggccagaatttgcagataaccatagaactcttctcctccatcaggcatggatttagcctcctttagttcctgcagtgacacaggagcctccaaataccaaattattatcaggcggtcttgggggaacctctgggc

SEQ ID NO: 21 - DNA, 1030 bp, Homo sapiens
  misc_feature: Anti-sense RNA complementary to human truncated TrkC exon 13B
aagcttcctcccctggaagacactcctttaatttaagggatttttttagtaccaaatcccgtgctgggcacctagtcgtacctcagtccacgaaatatatggaggcttgatgagtcttaaggtcttaagaaccaaaattggttaggggagggatctttactgcatacaaagacgtcaaaggaggtaactcaccatgtgaccttgggtaagacacttccccactctggacctcaggttcctcatatatatagtgagttgatgggactagatgatctctattgtccttcaagtttaatatcccatgattgtctatgtttgaaaagacccctggcagaagaggcagacatgggggaattaatggtcagtattaaaccccaagtgcaaaaaacatgggaaagtatttttcttaagtgcacaatgccagaaacagaaatccaggtttctcagctcctgtacccactctgctgtgcctgtagtggcagctggtttattttacgcataaaatctagatcctaagaatctcaccctggcattatagaaatggcacaagttagagtcagagaagaaagattcaccatcaccatgaactctgtcctgcacttttcctaacatatttgcccccattttccttcttggaccagaatgtctcaaaatggtgcccatcattgccattcagagtgtcattcctgggcctggatggatgtttcttctgacttgggagataatcctggacccaagaccctctttctggcttctaatatcccatcaaaaaaattcagctaattttcaagaggaaagcaatgtgacaaggcggggatgatgaggagatgagaggaggtggtggtgtttggttccttaaaatcttccttgccattgttagggcataaaccattggaaaaaccaaagctgttagctcactgaagacctgaagactacaacttctataaagagatatcattgtagcttctgaggatgaaatctcctttggagaccatgcagcatttgtgggccctggaaagatccgtgtcgg

SEQ ID NO: 22 - DNA, 1113 bp, Homo sapiens
  misc_feature: Anti-sense RNA complementary to human truncated TrkC exon 14B
aagcttgtcggaactgcagatctaaggccccaggaagagcctggagaaactctcctctgcaaggagttttctaatgtcacactcattgtgagccgttccagagagaagaggaagtagggggcaagtatgtcccaaaaaaggatgatttttccaccgaaaataaaaataaaaatgtaaagtgattggtttccattctttcctctgagacctaacttggtgccaatacttgagcctggctccccctcctcttaccgcgccgtctccacctctgcttaggtgctctctgtgactcttccccactggaggttgcagggcgacgaggtcactaagaggacagcatggtatcctgggagagccagggagaccccacgcttccaagcagggaaagtgaggctggagcacggctggacttcatcaggctttcttgtgatggttaggttgtttttgtaaagcaagagaatggctagtgtatttaaaagatgctcctggtgcacaggtttttaattctctttattggctctaaagatagtggcaggagctgcctcgtaggccgggaaaaaaggagcacagtgatgattggagggaaggggcaaccctgccagtggtggatgggggaaaacacccatctggtggtgttaaagggtgccggggcactggtggctcatgcagtttctacttagcatccctggatccaaagagaacaatgcctagagcttccaacctctcagagggcccagcctaccaaggtgacatcaaaacaaggaggcttaaaaggagtttttaaaagccatgacgtcctttgctgaaataaacattgacatcctcaacatagatgccatggttaagaggcttggaatgtccgggaaggcttattggattcaacataatttctctgaaacctataaaaaacaaaaagaaaaaaaaacagaaaaaagcaaaataaagtaaaaaccaaaacccccaaagaagatcaaaactaggaggggaaatagagagggagcttgaaaggggaagaactgtcagcaaaagggaaggataggaggaggggattaacttaaaaccctgcatggtcagagaaactggaaacactccattttgctgcagcctccactg